(19) United States
(12) Patent Application Publication
(10) Pub. No.: US 2025/0120683 A1
(43) Pub. Date: Apr. 17, 2025

(54) APPARATUS AND METHOD TO REDUCE PARASITIC LOSSES OF THE ELECTRICAL SYSTEM OF A SURGICAL INSTRUMENT

(71) Applicant: Cilag GmbH International, Zug (CH)

(72) Inventors: Nicholas J. Ross, Franklin, OH (US); Frederick E. Shelton, IV, Hillsboro, OH (US); Shane R. Adams, Lebanon, OH (US); Taylor W. Aronhalt, Loveland, OH (US)

(21) Appl. No.: 18/379,773

(22) Filed: Oct. 13, 2023

(51) Int. Cl. A61B 17/00; H02J 7/00; H02M 3/158; H02P 29/00

(52) U.S. Cl. CPC A61B 17/00; H02M 3/1582; A61B 2017/00398; H02J 7/007; H02J 2207/50; H02P 29/00; H02P 2201/09

(57) ABSTRACT

A surgical instrument comprises a motor, a regulator, a converter circuit, and a control circuit coupled to the regulator and the converter circuit. The control circuit is to couple either the regulator, or the converter circuit based on a pre-determined load voltage or current applied to the motor.

### TECHNICAL FIELD

The present disclosure is directed to surgical instruments and, in various arrangements, to surgical stapling and cutting instruments and staple cartridges for use therewith that are configured to staple and cut tissue.

### SUMMARY

The present disclosure provides a surgical instrument. The surgical instrument comprising: a motor, a low dropout (LDO) regulator, at least two converter circuits, and a control circuit coupled to the LDO regulator and the at least two converter circuits. The control circuit is to select a power source to apply to the motor from one of a battery, the LDO regulator, or at least one of the at least two converter circuits based on a load applied to the motor.

The present disclosure provides a surgical instrument. The surgical instrument comprising: a power supply and a control circuit comprising a plurality of electrical components. The control circuit is coupled to the power supply. The control circuit is to determine unused electrical components of the plurality of electrical components and sever a connection between the unused electrical components and the power supply.

The present disclosure provides a surgical instrument. The surgical instrument comprising: a power supply, a power accumulator, and a control circuit coupled to the power supply and the power accumulator. The control circuit is to charge the power accumulator, determine a parameter falls below a threshold, and discharge power from the power accumulator to increase power output.

### LISTING OF THE FIGURES

In the description, for purposes of explanation and not limitation, specific details are set forth, such as particular aspects, procedures, techniques, etc. to provide a thorough understanding of the present technology. However, it will be apparent to one skilled in the art that the present technology may be practiced in other aspects that depart from these specific details.

The accompanying drawings, where like reference numerals refer to identical or functionally similar elements throughout the separate views, together with the detailed description below, are incorporated in and form part of the specification, and serve to further illustrate aspects of concepts that include the claimed disclosure and explain various principles and advantages of those aspects.

The methods, devices, and systems disclosed herein have been represented where appropriate by conventional symbols in the drawings, showing only those specific details that are pertinent to understanding the various aspects of the present disclosure so as not to obscure the disclosure with details that will be readily apparent to those of ordinary skill in the art having the benefit of the description herein.

FIG. 1 illustrates an exploded view of an end effector and a shaft portion of a surgical stapling system, in accordance with the present disclosure.

FIG. 2 is a diagram illustrating components of a staple cartridge, in accordance with the present disclosure.

FIG. 3 is a diagram illustrating various components of a surgical stapling system, in accordance with the present disclosure.

FIG. 4 is a circuit, in accordance with the present disclosure.

FIG. 5 is a circuit, in accordance with the present disclosure.

FIG. 6 illustrates an example of a DC/DC converter type buck-boost converter circuit, in accordance with the present disclosure.

FIG. 7 illustrates example operational modes of an example DC/DC converter type buck-boost converter circuit such as the unloaded buck-boost converter circuit shown in FIG. 6, in accordance with the present disclosure.

FIG. 8 illustrates an example of a DC/DC converter type buck-boost converter circuit in accordance with the present disclosure.

FIG. 9 illustrates example power loss and efficiency at different load currents for a buck-boost DC/DC converter type buck-boost converter circuit such as the loaded buck-boost converter circuit shown in FIG. 8, in accordance with the present disclosure.

FIG. 10 illustrates a basic topology of a buck converter circuit that may be employed in accordance with the present disclosure.

FIG. 11 illustrates the output current of the buck converter circuit I_{OUT} shown in FIG. 10 in accordance with the present disclosure.

FIG. 12 illustrates the current I_{Q1} through the first transistor in FIG. 10, in accordance with the present disclosure.

FIG. 13 illustrates the current I_{Q2} through the second transistor in FIG. 10, in accordance with the present disclosure.

FIG. 14 is a graph of efficiency (%) as a function of output current (A) over a range of output currents of the buck converter circuit shown in FIG. 10 at discrete voltage values, in accordance with the present disclosure.

FIG. 15 is a graph of efficiency (%) as a function of load current (A) over a range of load currents for a LDO regulator, such as the LDO regulator (FIGS. 4 and 5) and a DC/DC converter type buck-boost converter circuits (FIGS. 6 and 8), in accordance with the present disclosure.

FIG. 16 illustrates a surgical system, in accordance with the present disclosure.

FIG. 17 illustrates an example implementation of a voltage boost converter circuit, in accordance with the present disclosure.

FIG. 18 is a graph illustrating efficiency (%) as a function of output current (A) for a DC/DC boost regulator, in accordance with the present disclosure.

FIG. 19 is a variable potentiometer, in accordance with the present disclosure.

FIG. 20 is a graph of drain current (ID) as a function of drain-to-source voltage (VDs) for switching a field effect transistor, in accordance with the present disclosure.

FIG. 21 is a graphical depiction of leakage current losses, in accordance with the present disclosure.

FIG. 22 is a graph of a battery discharge curve, in accordance with the present disclosure.

FIG. 23 is a super capacitor charging circuit, in accordance with the present disclosure.

FIG. 24 illustrates a motor drive circuit with boost circuit connections, in accordance with the present disclosure.

FIG. 25 is a vibration circuit to harvest energy from vibrations of the surgical stapling instrument (FIGS. 1-3), in accordance with the present disclosure.

FIG. 26 is a circuit to collect power from the movement of a drive bar of the surgical stapling instrument, in accordance with the present disclosure.

FIG. 27 is a circuit to collect power from the movement of a drive bar of the surgical stapling instrument, in accordance with the present disclosure.

FIG. 28 illustrates a graph of additional energy applied to the system from the circuit, in accordance with the present disclosure.

### DESCRIPTION

Applicant of the present application owns the following U.S. patent applications that were filed on even date herewith and which are each herein incorporated by reference in their respective entireties:
U.S. patent application, titled METHOD OF OPERATING A SURGICAL STAPLING INSTRUMENT; Attorney Docket No. END9484USNP2/220491-2M;
U.S. patent application, titled SURGICAL STAPLING SYSTEMS WITH ADAPTIVE STAPLE FIRING ALGORITHMS; Attorney Docket No. END9484USNP3/220491-3;
U.S. patent application, titled LEARNED TRIGGERS FOR ADAPTIVE CONTROL OF SURGICAL STAPLING SYSTEMS; Attorney Docket No. END9484USNP4/220491-4;
U.S. patent application, titled CONTROL CIRCUIT FOR ACTUATING MOTORIZED FUNCTION OF SURGICAL STAPLING INSTRUMENT UTILIZING INERTIAL DRIVE TRAIN PROPERTIES; Attorney Docket No. END9484USNP5/220491-5;
U.S. patent application, titled PROPORTIONATE BALANCING OF THE FUNCTION IMPACT MAGNITUDE OF BATTERY OUTPUT TO PEAK MOTOR CURRENT; Attorney Docket No. END9484USNP6/220491-6;
U.S. patent application, titled MOTOR OPTIMIZATION BY MINIMIZATION OF PARASITIC LOSSES AND TUNING MOTOR DRIVE CONFIGURATION; Attorney Docket No. END9484USNP7/220491-7;
U.S. patent application, titled SURGICAL TOOL WITH RELAXED FLEX CIRCUIT ARTICULATION; Attorney Docket No. END9484USNP9/220491-9;
U.S. patent application, titled WIRING HARNESS FOR SMART STAPLER WITH MULTI AXIS ARTICULATION; Attorney Docket No. END9484USNP10/220491-10;
U.S. patent application, titled SURGICAL SYSTEM WITH WIRELESS ARRAY FOR POWER AND DATA TRANSFER; Attorney Docket No. END9484USNP11/220491-11; and
U.S. patent application, titled SURGICAL STAPLE CARTRIDGE COMPRISING REPLACEABLE ELECTRONICS PACKAGE; Attorney Docket No. END9484USNP12/220491-12.

Applicant of the present application owns the following U.S. patent applications that were filed on even date herewith and which are each herein incorporated by reference in their respective entireties:
U.S. patent application, titled METHOD OF ASSEMBLING A STAPLE CARTRIDGE; Attorney Docket No. END9484USNP13/220491-13M;
U.S. patent application, titled CONTROL SURFACES ON A STAPLE DRIVER OF A SURGICAL STAPLE CARTRIDGE; Attorney Docket No. END9484USNP14/220491-14;
U.S. patent application, titled INTEGRAL CARTRIDGE STIFFENING FEATURES TO REDUCE CARTRIDGE DEFLECTION; Attorney Docket No. END9484USNP15/220491-15;
U.S. patent application, titled STAPLE CARTRIDGE COMPRISING WALL STRUCTURES TO REDUCE CARTRIDGE DEFLECTION; Attorney Docket No. END9484USNP16/220491-16;
U.S. patent application, titled PAN-LESS STAPLE CARTRIDGE ASSEMBLY COMPRISING RETENTION FEATURES FOR HOLDING STAPLE DRIVERS AND SLED; Attorney Docket No. END9484USNP17/220491-17;
U.S. patent application, titled STAPLE CARTRIDGE COMPRISING A SLED HAVING A DRIVER LIFT CAM; Attorney Docket No. END9484USNP18/220491-18;
U.S. patent application, titled SURGICAL STAPLE CARTRIDGES WITH SLEDS CONFIGURED TO BE COUPLED TO A FIRING DRIVER OF A COMPATIBLE SURGICAL STAPLER; Attorney Docket No. END9484USNP19/220491-19;
U.S. patent application, titled STAPLE CARTRIDGE COMPRISING A COMPOSITE SLED; Attorney Docket No. END9484USNP20/220491-20;
U.S. patent application, titled SURGICAL INSTRUMENTS WITH JAWAND FIRING ACTUATOR LOCKOUT ARRANGEMENTS LOCATED PROXIMAL TO A JAW PIVOT LOCATION; Attorney Docket No. END9484USNP21/220491-21;
U.S. patent application, titled SURGICAL INSTRUMENTS WITH LATERALLY ENGAGEABLE LOCKING ARRANGEMENTS FOR LOCKING A FIRING ACTUATOR; Attorney Docket No. END9484USNP22/220491-22;
U.S. patent application, titled DUAL INDEPENDENT KEYED LOCKING MEMBERS ACTING ON THE SAME DRIVE MEMBER; Attorney Docket No. END9484USNP23/220491-23;
U.S. patent application, titled ADJUNCTS FOR USE WITH SURGICAL STAPLING INSTRUMENTS; Attorney Docket No. END9484USNP24/220491-24;
U.S. patent application, titled ADJUNCTS FOR USE WITH SURGICAL STAPLING INSTRUMENTS; Attorney Docket No. END9484USNP25/220491-25;
U.S. patent application, titled JAW CONTROL SURFACES ON A SURGICAL INSTRUMENT JAW; Attorney Docket No. END9484USNP26/220491-26;
U.S. patent application, titled ZONED ALGORITHM ADAPTIVE CHANGES BASED ON CORRELATION OF COOPERATIVE COMPRESSION CONTRIBUTIONS OF TISSUE; Attorney Docket No. END9484USNP27/220491-27;
U.S. patent application, titled STAPLE CARTRIDGES COMPRISING TRACE RETENTION FEATURES; Attorney Docket No. END9484USNP29/220491-29; and
U.S. patent application, titled STAPLE CARTRIDGES COMPRISING STAPLE RETENTION FEATURES; Attorney Docket No. END9484USNP30/220491-30.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the described and illustrated embodiments are non-limiting examples, and thus it can be appreciated that the specific structural and functional details disclosed herein may be representative and illustrative. Variations and changes may be made without departing from the scope of the claims.

Various methods, instruments, and systems are provided for performing surgical procedures. Various surgical systems disclosed herein include working portions that can be inserted into a body in any way, such as through a natural orifice, through an incision or puncture hole formed in tissue, etc. The working portions or end effector portions can be inserted directly into a patient's body or can be inserted through an access device that has a working channel. As the present Detailed Description proceeds, it will be understood that the various unique and novel arrangements of the various forms of surgical systems disclosed herein may be effectively employed in connection with robotically-controlled surgical systems and/or hand-held surgical systems. Various robotic systems, instruments, components and methods are disclosed in U.S. patent application Ser. No. 13/118,241, entitled SURGICAL STAPLING INSTRUMENTS WITH ROTATABLE STAPLE DEPLOYMENT ARRANGEMENTS, which is incorporated by reference herein in its entirety.

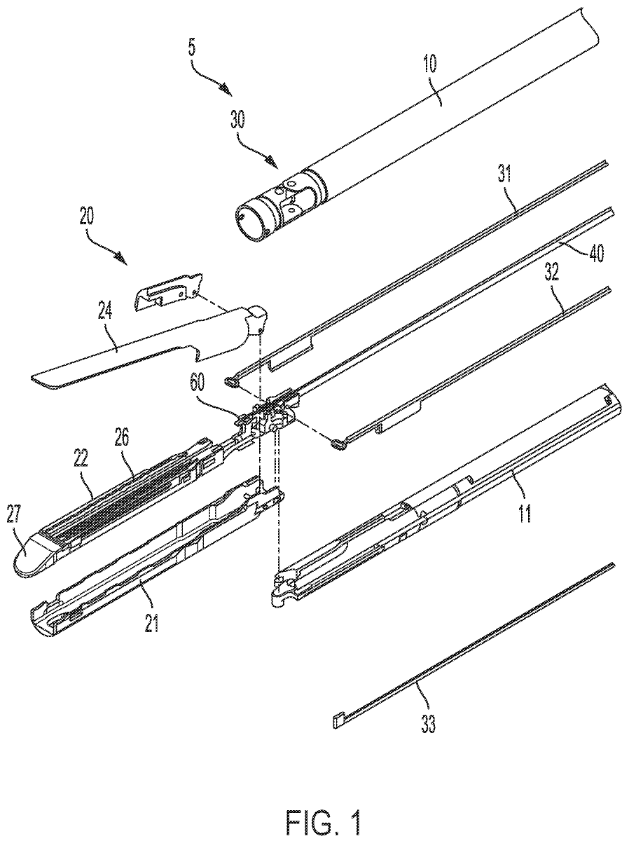

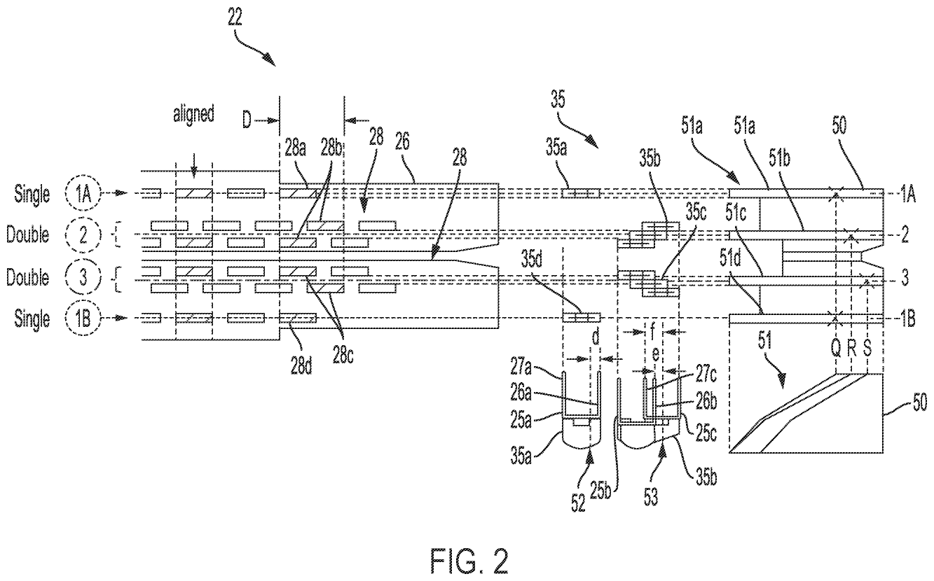

Referring to FIGS. 1-3, a surgical stapling instrument 5 includes a shaft 10 and an end effector 20 extending from the shaft 10. The end effector 20 includes a first jaw and a second jaw. The first jaw defines a channel 21 and a staple cartridge 22 removably positionable in the channel 21. However, other embodiments are envisioned in which a staple cartridge is not removable, or at least readily replaceable, from the first jaw. The second jaw includes an anvil 24 configured to deform the staples 25 (See FIG. 2) ejected from the staple cartridge 22. The second jaw is pivotable relative to the first jaw about a closure axis to transition the end effector 20 between an open configuration and a closed configuration. Other embodiments are envisioned in which the first jaw is pivotable relative to the second jaw.

The surgical stapling instrument 5 further includes an articulation joint 30 configured to permit the end effector 20 to be rotated, or articulated, relative to the shaft 10. The end effector 20 is rotatable about an articulation axis extending through the articulation joint 30. Other embodiments are envisioned which do not include an articulation joint. In the illustrated example, cooperating articulation rods 31, 32 are configured to articulate the end effector 20 relative to the shaft 10 about an articulation joint 30. The surgical stapling instrument 5 further includes an articulation lock bar 33 the selectively prevents the articulation of the end effector 20.

The staple cartridge 22 includes a cartridge body 27 with a proximal end, a distal end, and a deck 26 extending between the proximal end and the distal end. In use, the staple cartridge 22 is positioned on a first side of the tissue to be stapled and the anvil 24 is positioned on a second side of the tissue. In accordance with the present disclosure, the anvil 24 can be moved toward the staple cartridge 22 to compress and clamp the tissue against the deck 26. Additionally, the staple cartridge 22 can be moved relative to the anvil 24 or, alternatively, both the staple cartridge 22 and the anvil 24 can be moved to compress and clamp the tissue.

Further to the above, a drive shaft 40 is movable distally to motivate a firing beam 60 to transition the end effector 20 toward the closed configuration, thereby compressing the tissue. In the illustrated example, the firing beam 60 is in the form of an I-beam that includes a first cam and a second cam configured to engage the first and second jaws, respectively. As the firing beam 60 is advanced distally, the first cam and the second cam can control the distance, or tissue gap, between the deck of the staple cartridge 22 and the anvil 24. In the illustrated example, the firing beam 60 motivates a sled 50 to deploy the staples 25 from the staple cartridge 22. In accordance with the present disclosure, a separate closure mechanism, e.g. a closure tube, can be employed to transition the end effector 20 toward the closed configuration. The firing beam 60 may or may not include the first and second cams. Additionally, the firing beam 60 may in the form of an E-beam with first, second, and third cams. Further, in accordance with the present disclosure, the firing beam 60 and the closure tube can cooperatively effect closure of the end effector 20. Alternatively, the firing beam 60 may only effect deployment of the staples 25.

In accordance with the present disclosure, as illustrated in FIG. 1, the firing beam 60 can include a knife configured to incise the tissue captured intermediate the staple cartridge 22 and the anvil 24. It is desirable for the knife to be positioned at least partially proximal to the ramped surfaces such that the staples are ejected ahead of the knife. More details about alternative embodiments of surgical stapling systems, suitable for use with the present disclosure, are disclosed in U.S. patent application Ser. No. 15/385,887 entitled METHOD FOR ATTACHING A SHAFT ASSEMBLY TO A SURGICAL INSTRUMENT AND, ALTERNATIVELY, TO A SURGICAL ROBOT, and U.S. patent application Ser. No. 16/209,416, entitled METHOD OF HUB COMMUNICATION, PROCESSING, DISPLAY, AND CLOUD ANALYTICS, which are hereby incorporated by reference herein in their entireties.

The staples 25 removably stored in the cartridge body 27 can be deployed into the tissue. The cartridge body 27 includes staple cavities 28 defined therein wherein staples 25 are removably stored in the staple cavities 28. The staple cavities 28 are arranged in longitudinal rows. In the illustrated example, three rows of staple cavities 28 are positioned on a first side of a longitudinal slot 29 and three rows of staple cavities 28 are positioned on a second side of the longitudinal slot. Other arrangements of staple cavities 28 and staples 25 are possible.

The staples 25 are supported by staple drivers 35 in the cartridge body 27. The staple drivers 35 are movable between a first, or unfired position, and a second, or fired, position to eject the staples 25 from the staple cavities 28. The staple drivers 35 are movable between their unfired positions and their fired positions by a sled 50 that includes ramped surfaces 51 configured to slide under the staple drivers 35 and lift the staple drivers 35, and the staples 25 supported thereon, toward the anvil 24. In the illustrated example, the distal movement of the drive shaft 40 causes the sled 50 to move distally within the staple cartridge 22 to deploy the staples 25.

Referring primarily to FIG. 2, the sled 50 includes a first ramped surface 51a, a second ramped surface 51b, a third ramped surface 51c, and a fourth ramped surface 51d configured to engage a first staple drive 35a, a second staple driver 35b, a third staple driver 35c, and a fourth staple driver 35d, respectively, along a staple-forming distance (D) to deploy staples 25 from corresponding staple cavities 28a, 28b, 28c, 28d for forming against corresponding forming pockets in the anvil 24. In the illustrated example, the staple drivers 35b, 35c are double drivers, while the staple drivers 35a, 35d are single drivers. Double drivers support two staples in two separate staple cavities, while single drivers support a single staple in a single staple cavity.

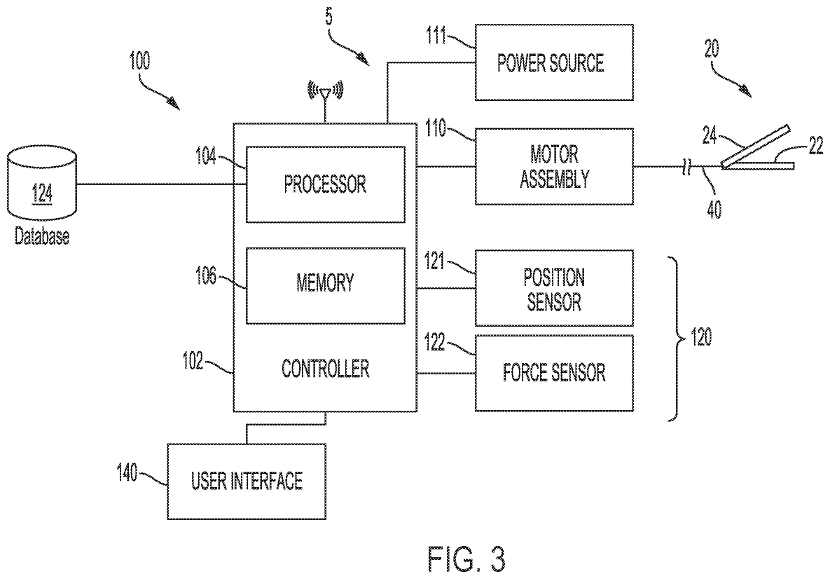

FIG. 3 is a block diagram illustrating one exemplification of the surgical stapling instrument 5. Various components of the surgical stapling instrument 5 communicate with a control circuit 100. Such components may receive signals from and/or transmit signals to the control circuit 100. Such signals include command signals, status signals, sensor signals, and/or any other suitable signals. The control circuit 100 can be configured to implement various methods described herein with the aid of various components of the surgical stapling system in communication with the control circuit 100. In the illustrated example, the control circuit 100 includes a controller 102 comprising one or more processors 104 (e.g., microprocessor, microcontroller) coupled to at least one memory circuit 106. The memory circuit 106 stores machine executable instructions that when executed by the processor 104, cause the processor 104 to execute machine instructions to implement various processes described herein. The processor 104 may be any one of a number of single or multi-core processors known in the art. The memory circuit 106 may comprise volatile and non-volatile storage media. The processor 104 may include an instruction processing unit and an arithmetic unit. The instruction processing unit may be configured to receive instructions from the memory circuit 106. The control circuit 100 can include a combinational logic circuit and/or a sequential logic circuit. The processor 104 is in communication with a database 124 to store various information associated with the surgical stapling instrument 5. In accordance with the present disclosure, the database 124 can store and identifier of a staple cartridge, or other component of the end effector 20.

In accordance with the present disclosure, the control circuit 100 can be configured to communicate with a motor assembly 110 that includes a motor and a motor controller, for example. The motor assembly may generate rotational motion to effect a translating motion of the drive shaft 40. The control circuit 160 may generate a motor set point signal. The motor set point signal may be provided to a motor controller. The motor controller may comprise one or more circuits configured to provide a motor drive signal to a motor to drive the motor as described herein. The motor may be a brushed DC electric motor. For example, the velocity of the motor may be proportional to the motor drive signal. Alternatively, the motor may be a brushless DC electric motor and the motor drive signal may comprise a PWM signal provided to one or more stator windings of the motor. Also, the motor controller may be omitted, and the control circuit 100 may generate the motor drive signal directly. The position, movement, displacement, and/or translation of the drive shaft 40, the firing beam 60 and/or the sled 50 (collectively referred to herein as the “firing assembly”) can be measured/monitored by the control circuit 100 based on input from one or more sensors 120.

The motor assembly 110 may be powered by a power source 111 that in one form may comprise a removable power pack. The power pack may include a housing configured to support a plurality of batteries that may each include, for example, a Lithium Ion (“LI”) or other suitable battery, and may be connected in series, for example. The power source 111 may be replaceable and/or rechargeable. Other power sources are contemplated by the present disclosure.

The sensors 120 may include a position sensor 121 configured to sense a position, movement, displacement, and/or translation of one or more components of the firing assembly such as, for example, the drive shaft 40, the firing beam 60 and/or the sled 50. The position sensor 121 may include any type of sensor that is capable of generating position data that indicate a position of the firing assembly. In accordance with the present disclosure, the position sensor 121 may include an encoder configured to provide a series of pulses to the control circuit 100 as the firing assembly translates distally and proximally. The control circuit 100 may track the pulses to determine the position, movement, displacement, and/or translation of the components of the firing assembly. Other suitable position sensors may be used, including, for example, a proximity sensor. Other types of position sensors may provide other signals indicating motion of the firing assembly. Where the motor is a stepper motor, the control circuit 100 may track the position of components of the firing assembly by aggregating the number and direction of steps that the motor has been instructed to execute. The sensors 120 may be located in the end effector 20 or at any other portion of the surgical stapling instrument 5.

Various sensors 120 may be adapted to measure various other parameters such as gap distance versus time, tissue compression versus time, and anvil strain versus time. The sensors 120 may comprise a magnetic sensor, a magnetic field sensor, a strain gauge, a pressure sensor, a force sensor, an inductive sensor such as an eddy current sensor, a resistive sensor, a capacitive sensor, an optical sensor, and/or any other suitable sensor for measuring one or more parameters of the end effector 20. The one or more than one sensor 120 may be sampled in real time during a clamping operation by the processor 104 of the control circuit 100. The control circuit 100 receives real-time sample measurements to provide and analyze time-based information and assess, in real time, a measured parameter such as, for example, force and/or position parameters.

The one or more than one sensor 120 may comprise a strain gauge, such as a micro-strain gauge, configured to measure the magnitude of the strain in the anvil 24 during a clamped condition. The strain gauge provides an electrical signal whose amplitude varies with the magnitude of the strain. The sensors 120 may comprise a pressure sensor configured to detect a pressure generated by the presence of compressed tissue between the anvil 24 and the staple cartridge 22. The sensors 120 may be configured to detect impedance of a tissue section located between the anvil 24 and the staple cartridge 22 that is indicative of the thickness and/or fullness of tissue located therebetween.

The sensors 120 may include a force sensor 122 configured to measure forces associated with firing and/or closure conditions. For example, the force sensor 122 can be at an interaction point between a closure tube and the anvil 24 to detect the closure forces applied by a closure tube to the anvil 24. The forces exerted on the anvil 24 can be representative of the tissue compression experienced by the tissue section captured between the anvil 24 and the staple cartridge 22. The force sensor 122 can be positioned at various interaction points along the closure drive system to detect the closure forces applied to the anvil 24.

Similarly, a force sensor 122 can be at an interaction point between components of the firing assembly to detect the firing forces applied by the firing assembly to advance the firing beam 60 and the sled 50 to deploy the staples into tissue and cut the tissue. The measured forces represent a firing load experienced by the firing assembly. Alternatively, or additionally, a current sensor can be employed to measure the current drawn by the motor of the motor assembly 110. The force required to advance the firing assembly corresponds to the current drawn by the motor. The measured force can be converted to a digital signal and provided to the control circuit 100.

Further to the above, the surgical stapling instrument 5 includes a user interface 140 having an input device (e.g., a capacitive touchscreen or a keyboard) for receiving inputs from a user and an output device (e.g., a display screen) for providing outputs to a user. Outputs can include data from a query input by the user, suggestions for products or mixes of products to use in a given procedure, and/or instructions for actions to be carried out before, during, or after surgical procedures. The user interface 140 can be in communication with the control circuit 100, as illustrated in FIG. 3.

During a surgical stapling procedure, a clinician may operate a surgical stapling instrument 5 to fire sequentially multiple staple cartridges along a selected tissue resection line to achieve a clinical outcome. For example, in a stomach resection procedure, the clinician may sequentially fire staple cartridges of different characteristics (e.g., size, color, type, length, staple height, staple diameter, staple size) along a selected tissue resection line to remove a portion of the stomach. In operation, the staple cartridges fire along the tissue resection line in an end-to-end arrangement.

The clinician may examine the tissue selected for resection using any suitable imaging technique such as, for example, x-ray, registered magnetic resonance imaging (MRI), and/or computerized tomography (CT) scan. The clinician may then select a suitable tissue resection line, and staple cartridges for sequential firing along the selected tissue resection line. Visual examination, however, has its limitations, and a tissue response to stapling can vary depending on many factors including, for example, patient age, tissue health, and/or tissue type. Moreover, tissue thickness and/or stiffness may vary along the selected tissue resection line, resulting in unexpected tissue responses.

Various methods, devices, and systems are provided for adaptively adjusting operational parameters of the surgical stapling instrument 5 during a staple cartridge firing based on a tissue response in an earlier phase/zone of the staple cartridge firing. Moreover, various methods, devices, and systems are provided for adaptively adjusting operational parameters of the surgical stapling instrument 5 during a staple cartridge firing based on a tissue response in one or more previous staple cartridge firings in a surgical procedure involving multiple sequential firings.

In accordance with the present disclosure, the processor 104 can execute various program instructions, which can be stored in a memory circuit such as the memory circuit 106, to implement various algorithms associated with firing a staple cartridge by the surgical stapling instrument 5. Such algorithms, e.g., thresholds, limits, triggers, conditions, pauses, and/or wait time, can be adjusted based on information learned from a tissue response in an earlier phase/zone of the staple cartridge firing, and/or based on a tissue response in one or more previous staple cartridge firings in a surgical procedure involving multiple sequential firings, as described in more detail below.

Parasitic losses occur within the circuitry of a surgical instrument. Parasitic losses can occur when power is consumed even when the device is powered off. Additionally, parasitic losses can occur when energy is lost during the powering of components in an electrical circuit. Momentary overcoming of the parasitic losses within the surgical instrument allows the instrument to minimize the diversion of power away from the motor and increase the power of the motor. The three main contributors to parasitic losses in power semiconductors are conduction, switching, and blocking (also known as reverse leakage). Conduction losses are the product of current flowing through an electrical component. The ripple current affects the power loss.

In accordance with the present disclosure, the surgical instrument may comprise a control circuit that can actively reduce its draw of power to enable the motor to use the full battery output. Battery powered surgical instruments have control electronics that are capable of operating a motor control circuit while reducing the power draw of the control electronics from the primary power source to such a level that the motor is capable of utilizing nearly all of the outputted power of the battery. The control electronics can have an integrated power accumulator and/or a boost circuit capable of providing the control circuit with a voltage level that is different from the voltage of the primary battery pack. Additionally, the control electronics can be selectively severable from the output power of the primary battery pack temporarily and re-coupleable. The electrical components can be temporarily removed from the primary power source.

In addition, optimization of power supply efficiency for a rated output current optimizes overall circuit efficiency and reduces circuit losses. A power supply with multiple converter circuits may be capable of specifying different loading conditions (voltage and current requirements). The converter circuits can be any of a buck, boost, or buck-boost converter circuit. LDO regulators are more efficient than power supplies at low loading situations. An LDO voltage regulator, for example, optimizes voltage output based on pre-determined loading conditions. The surgical device may switch from a power supply to an LDO regulator during low loads to increase battery life. In switching from one power source to another, additional capacitance holds the voltage rail until the switch is complete.

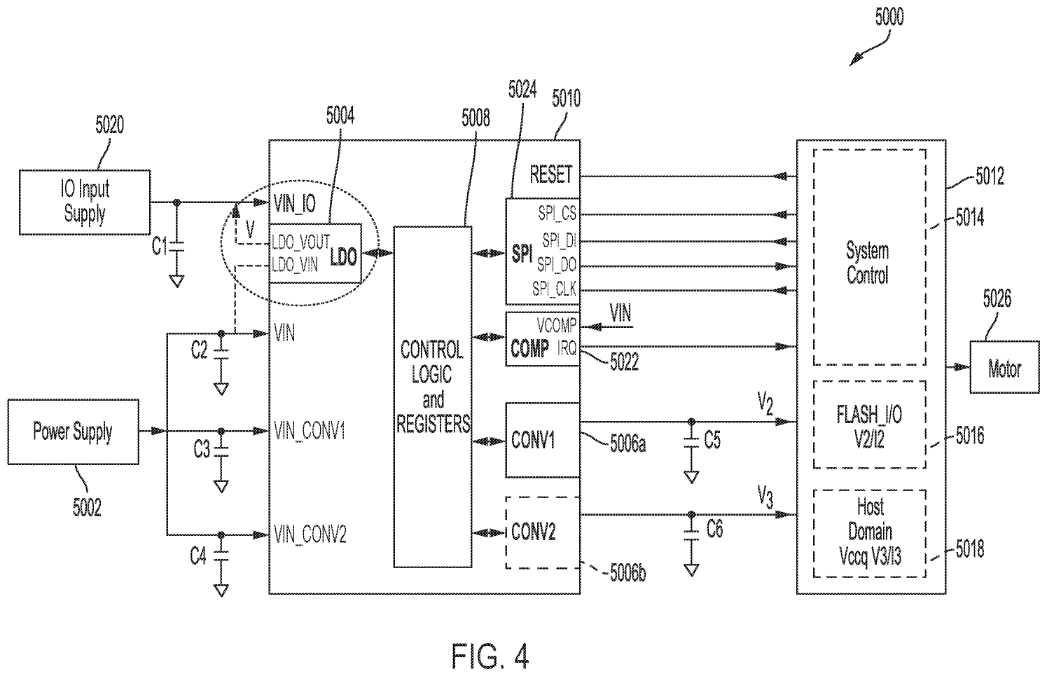

FIG. 4 is a circuit 5000, in accordance with the present disclosure. The circuit 5000 is part of a surgical instrument. The circuit 5000 can be part of the surgical stapling instrument 5 shown in FIGS. 1 and 3, for example. With reference now to FIGS. 1, 3, and 4, the surgical stapling instrument 5 may comprise the circuit 5000. The circuit 5000 comprises a power management circuit 5010 coupled to a control circuit 5012 to reduce or overcome parasitic losses of the surgical stapling instrument 5. The power management circuit 5010 is a power management circuit comprising two configurable, high-efficiency buck regulators for supplying variable voltages. The circuit 5000 reduces the electrical system parasitic losses and running losses including temporarily removing control electronics from the primary power source. The circuit 5000 can enable the motor 5026 to access the full capacity of the power source (e.g., a battery).

The power management circuit 5010 receives input power from a power supply 5002 and an Input/Output (IO) power supply 5020. In the example illustrated in FIG. 4, the power management circuit 5010 comprises a LDO regulator 5004, a plurality of converter circuits 5006a, 5006b, and a control logic and register circuit 5008 coupled between the LDO regulator 5004 and the plurality of converter circuits 5006a, 5006b. The converter circuits 5006a, 5006b can be any of a buck, boost, or buck-boost converter circuit. The power management circuit 5010 may comprise a single converter circuit 5006a. The power management circuit 5010 also comprises a comparator 5022 and a serial peripheral interface 5024 (SPI). The power management circuit 5010 operates cooperatively with the control circuit 5012 to optimize the supply voltage to the motor 5026 and low-power conditions and power saving modes via the SPI 5024. The power management circuit 5010 also supports the LDO regulator 5004 and a programmable interrupt comparator 5022 to monitor VIN. The motor 5026 is coupled to the power supplies of the power management circuit 5010 through the control circuit 5012.

The control circuit 5012 comprises a system control circuit 5114, a flash I/O circuit 5016, and a host domain circuit 5018. The control circuit 5012 communicates with the power management circuit 5010 through the SPI 5024 to control the control logic and register circuit 5008 to select the output voltages V2, V3 supplied by the converter circuit 5006a, 5006b, respectively. In accordance with the present disclosure, the output voltage V2 may be in a range from 1.1V to 3.6V at 1A and the output voltage V3 is in a range from 0.7V to 1.335V at 1A. Further, in accordance with the present disclosure, the LDO regulator 5004 may output voltage V1 of approximately 3.0V at 250 mA. The control circuit 5012 may comprise additional control electronics (not shown) that draw power from the power supply 5002. The control circuit 5012 can be one embodiment of the control circuit 100 shown in FIG. 3.

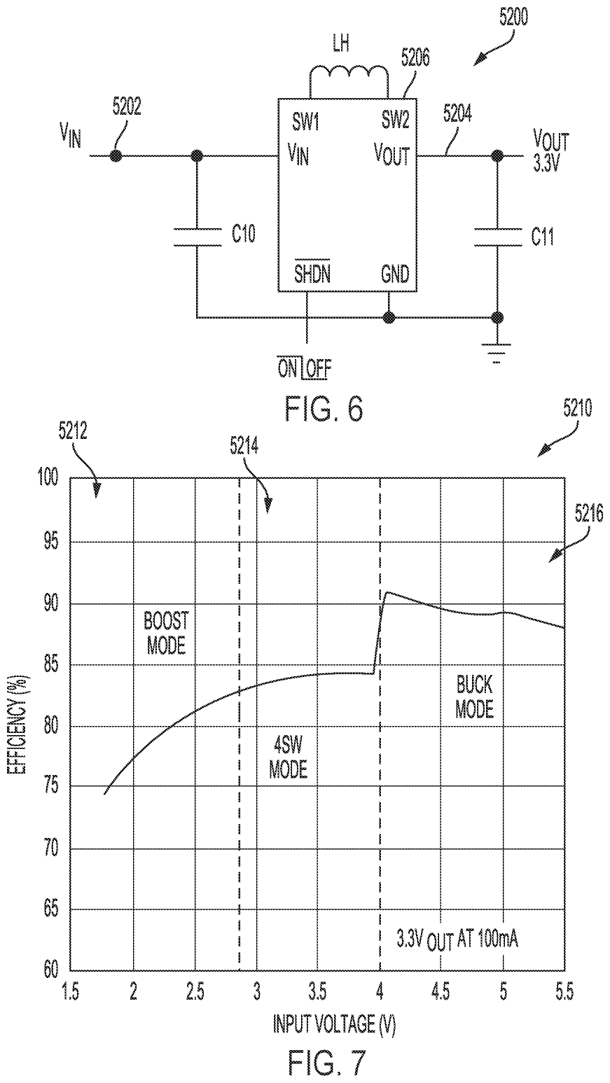

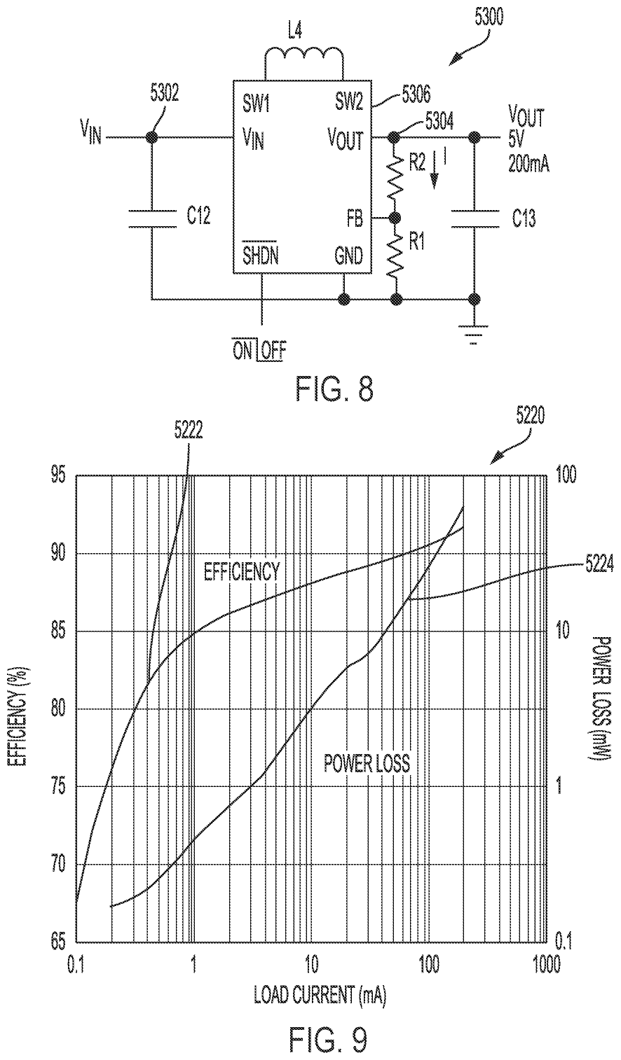

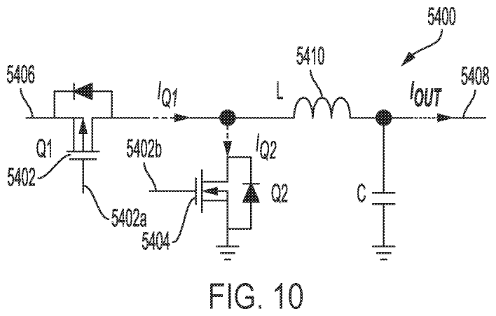

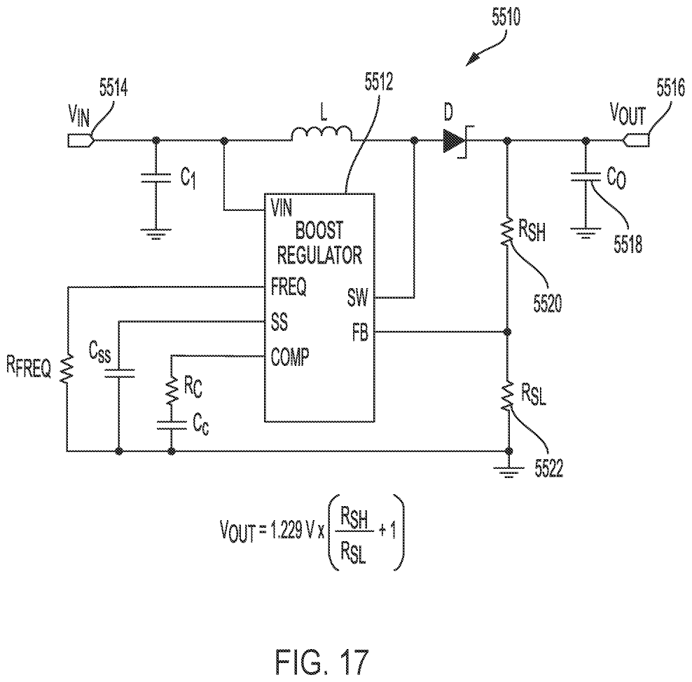

With continued reference to FIGS. 1, 3, and 4, the control circuit 5012 measures the load to the motor 5026. The control circuit 5012 measures the load to the motor 5026 with a voltage monitor (e.g., voltage sensor or voltmeter) and/or a current monitor (e.g., current sensor or ammeter) coupled between the motor 5026 and the power supply. The control circuit 5012 can couple a power source from one of the power supply 5002, the LDO regulator 5004, or the plurality of converter circuits 5006a, 5006b based on the load to the motor 5026 and can cause the motor 5026 to receive power from the selected source. The control circuit 5012 can couple either the regulator 5004 or the converter circuit 5006a based on a pre-determined voltage or current load applied to the motor. The motor 5026 in cooperation with the shaft 10 and the end effector 20 causes a surgical effect. The control circuit 5012 can determine the power source for the motor 5026 based in part on the load to the motor 5026. The selected power source is one of the power supply 5002, the LDO regulator 5004, or the plurality of converter circuits 5006a, 5006b. The load can be one of or both of voltage (V) load of the motor 5026 or a current load (I) of the motor 5026. In accordance with the present disclosure, the converter circuits 5006a, 5006b can be any of a buck (shown in FIG. 10), boost (shown in FIG. 17), or buck-boost (shown in FIGS. 6 and 8) converter circuit.

The control circuit 5012 stores the operating range of the power supply 5002, the LDO regulator 5004, and the plurality of converter circuits 5006a, 5006b. In accordance with the present disclosure, the expected operating range of the power supply 5002, the LDO regulator 5004, and the plurality of converter circuits 5006a, 5006b may dictate the selection of the power source. The operating range includes at least one of the voltage (V) and current (I) output of each power source.

The control circuit 5012 stores the efficiencies of each selectable power source, for example, the power supply 5002, the LDO regulator 5004, and the plurality of converter circuits 5006a, 5006b. In addition, the control circuit 5012 stores the voltage (V) and current (I) output of each power source. The control circuit 5012 may determine the power source based in part on the voltage (V) and current (I) output of each of the selectable power sources.

In accordance with the present disclosure, the control circuit 5012 can couple the power sources of the power management circuit 5010, such as for example, the LDO regulator 5004 and the plurality of converter circuits 5006a, 5006b selected by the control circuit 5012, to the motor 5026. Where at least one of the converter circuits 5006a, 5006b is a boost converter circuit, the control circuit 5012 can be configured to couple the boost converter circuit to the motor 5026 based on the voltage load at the motor 5026 exceeding the pre-determined voltage load. Additionally, where at least one of the converter circuits 5006a, 5006b is a boost converter circuit, the control circuit 5012 can be configured to determine a voltage sag at the motor 5026 and can couple the voltage boost converter circuit to the motor 5026. Alternatively, where at least one of the converter circuits 5006a, 5006b is a buck converter circuit, the buck converter circuit 5012 can be selected based on the current exceeding the pre-determined current load. The regulator 5004 can be a low dropout (LDO) regulator, and the control circuit 5012 can couple the motor 5026 to the LDO regulator based on the voltage load of the motor 5026 being below the pre-determined voltage load.

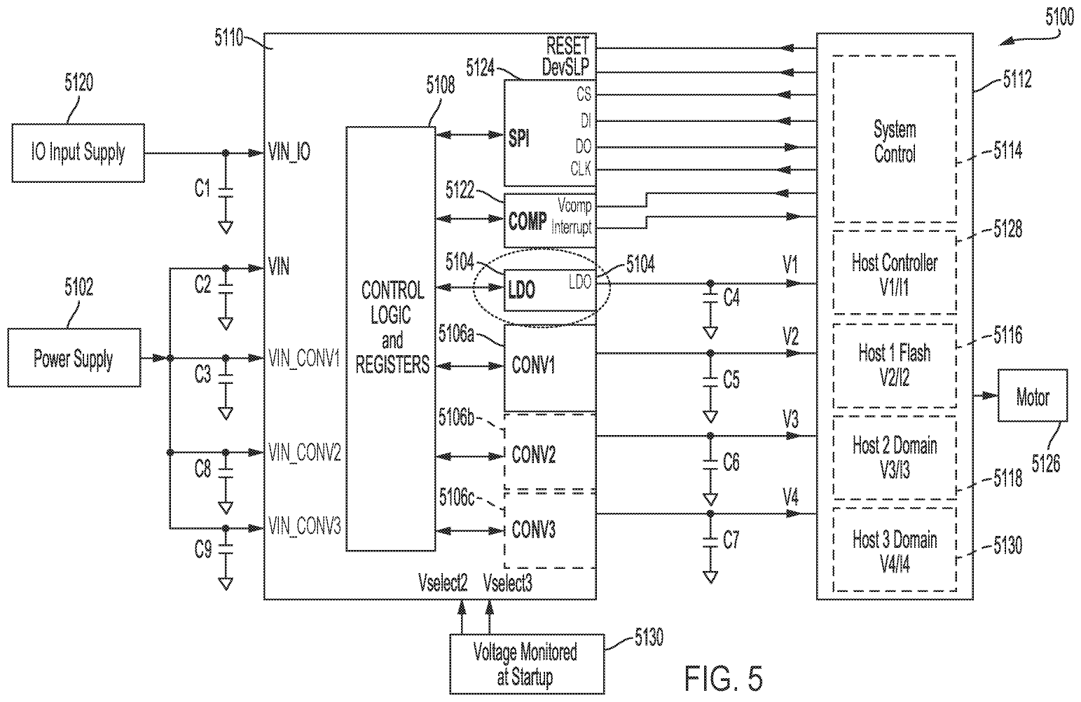

FIG. 5 is a circuit 5100 in accordance with the present disclosure. The circuit 5100 can be part of the surgical stapling instrument 5 shown in FIGS. 1 and 3, for example. With reference now to FIGS. 1, 3, and 5, the surgical stapling instrument 5 may comprise the circuit 5100. The circuit comprises a power management circuit 5110 coupled to a control circuit 5112 to reduce or overcome parasitic losses of the surgical stapling instrument 5. The power management circuit 5110 is an advanced power management unit comprising three configurable, high-efficiency buck regulators for supplying variable voltages. The circuit 5100 reduces the electrical system parasitic losses and running losses including temporarily removing control electronics from the primary power source. The circuit 5100 can enable the motor 5126 to access the full capacity of the power source (e.g., a battery).

Similar to the power management circuit 5010 described in connection with FIG. 4, the power management circuit 5110 shown in FIG. 5 receives input power from a power supply 5102 and an I/O power supply 5120. The power management circuit 5110 comprises a LDO regulator 5104, a plurality of converter circuits 5106a, 5106b, 5106c, and a control logic and register circuit 5108 coupled between the LDO regulator 5104 and the plurality of converter circuits 5106a, 5106b, 5106c. The converter circuits 5106a, 5106b, 5106c can be any of a buck (shown in FIG. 10), boost (shown in FIG. 17), or buck-boost (shown in FIGS. 6 and 8) converter circuit. The power management circuit 5510 may comprise a single converter circuit 5106a. The power management circuit 5110 also comprises a comparator 5122 and a SPI 5124. The power management circuit 5110 operates cooperatively with the control circuit 5112 to optimize the supply voltage to the motor 5126 and low-power conditions and power saving modes via the SPI 5124. The power management circuit 5110 also supports the LDO regulator 5104 and a programmable interrupt comparator 5122 to monitor VIN. The motor 5126 is coupled to the power supplies of the power management circuit 5110 through the control circuit 5112.

The control circuit 5112 comprises a system control circuit 5114, a host controller 5128, a host-1 flash I/O circuit 5116, a host-2 domain circuit 5118, and a host-3 domain circuit 5130. The control circuit 5112 communicates with the power management circuit 5110 through the SPI 5124 to control the control logic and register circuit 5108 to select the output voltages V2, V3, V4 supplied by the converter circuit 5106a, 5106b, 5106c, respectively. In accordance with the present disclosure, the output voltage V2 may be in a range from 1.1V to 3.6V at 1.6 A, the output voltage V3 is in a range from 1.1V to 3.6V at 1 A, and the output voltage V3 is in a range from 0.7V to 1.335V at 1A. Further, in accordance with the present disclosure, the LDO regulator 5104 may output voltage V1 at approximately 1.2V to 3.1V at up to 250 mA. The control circuit 5112 may comprise additional control electronics (not shown) that draw power from the power supply 5102. The control circuit 5112 can be one embodiment of the control circuit 100 shown in FIG. 3.

With continued reference to FIGS. 1, 3, and 5, the control circuit 5112 measures the load to the motor 5126. The control circuit 5112 measures the load to the motor 5126 with a voltage monitor (e.g., voltage sensor or voltmeter) and/or a current monitor (e.g., current sensor or ammeter) coupled between the motor 5016 and the power supply. The control circuit 5112 can select a power source from one of the power supply 5102, the LDO regulator 5104, or the plurality of converter circuits 5106a, 5106b, 5106c based on the load and can cause the motor to receive power from the selected source. Additionally, the control circuit 5012 can couple either the regulator 5004 or the converter circuit 5006a based on a pre-determined voltage or current load applied to the motor. The motor 5126 in cooperation with the shaft 10 and the end effector 20 causes a surgical effect. The control circuit 5112 can determine the power source for the motor 5126 based in part on the load to the motor 5126. The selected power source is one of the power supply 5102, the LDO regulator 5104, or the plurality of converter circuits 5106a, 5106b, 5106c. The load can be one of or both of voltage (V) load of the motor 5126 or a current (I) load of the motor. The converter circuits 5106a, 5106b, 5106c can be any of a buck, boost, or buck-boost converter circuit.

The control circuit 5112 stores the operating range of the power supply 5102, the LDO regulator 5104, and the plurality of converter circuits 5106a, 5106b, 5106c. In accordance with the present disclosure, the selection of the power source may be based on the expected operating range of the power supply 5102, the LDO regulator 5104, and the plurality of converter circuits 5106a, 5106b, 5106c. The operating range includes at least one of the voltage and current output of each power source.

The control circuit 5112 stores the efficiencies of each selectable power source, for example, the power supply 5102, the LDO regulator 5104, and the plurality of converter circuits 5106a, 5106b, 5106c. In addition, the control circuit 5112 stores the voltage and current output of each power source. The control circuit 5112 can determine the power source based in part on the voltage (V) and current (I) output of each of the selectable power sources.

In accordance with the present disclosure, the control circuit 5112 can couple the power sources of the power management circuit 5110, such as for example the LDO 5104 and the plurality of converter circuits 5106a, 5106b, 5106c selected power source by the control circuit 5112, to the motor 5126. Where at least one of the converter circuits 5106a, 5106b, 5106c is a boost converter circuit, the control circuit 5112 can be configured to couple the boost converter circuit to the motor 5126 based on the voltage load at the motor 5126 exceeding the pre-determined voltage load. Further, where at least one of the converter circuits 5106a, 5106b, 5106c is a boost converter circuit, the control circuit 5112 can be configured to determine a voltage sag at the motor 5126 and couple the voltage boost converter circuit to the motor 5126. Where at least one of the converter circuits 5106a, 5106b, 5106c is a buck converter circuit, the buck converter circuit 5112 may be selected based on the current exceeding the pre-determined current load. The regulator 5104 can be a low dropout (LDO) regulator, and the control circuit 5112 may couple the motor 5126 to the LDO regulator based on the voltage load of the motor 5126 being below the pre-determined voltage load.

The converter circuits 5006a, 5006b described in connection with FIG. 4 and the converter circuits 5106a, 5106b, 5106c described in connection with FIG. 5 can be implemented as the buck-boost converter circuits 5200, 5300 shown in FIGS. 6 and 8, respectively. The buck-boost converter circuits 5200, 5300 are described hereinbelow. FIG. 7 is graphical representation of the operating modes of the control circuit 100, 5012, 5112 shown in FIGS. 3-5 selected based on the efficiencies shown in FIG. 9.

FIGS. 6 and 8 illustrate examples of DC/DC converter type buck-boost converter circuits 5200, 5300. The buck-boost converter circuits 5200, 5300 operate from input voltages above, below, or equal to the output voltage. The buck-boost converter circuit 5200 may comprise an input 5202 from a voltage source (not shown) at a first voltage level, a buck-boost DC/DC converter 5206, and an output 5204 at a second voltage level. The buck-boost converter circuit 5200, 5300 can be any one of the converter circuits (5006a, 5006b, 5106a, 5106b, 5106c) of FIGS. 4 and 5

In accordance with the present disclosure, the output 5204 can be coupled to a motor (e.g., the motor 5026, 5126 shown in FIGS. 4-5). The buck-boost converter circuit 5200 outputs the second voltage level to the motor. The second voltage level is controllable by the control circuit (e.g., control circuit 100, 5012, 5112 shown in FIGS. 3-5). The control circuit controls the second voltage level applied to the motor. The first voltage level can be different from the second voltage level.

In accordance with the present disclosure, the buck-boost converter circuit 5300 shown in FIG. 8 can be similar to the buck-boost converter circuit 5200 shown in FIG. 6. The buck-boost converter circuit 5300 comprises an input 5302 from a voltage source VIN at a first voltage level, a DC/DC converter type buck-boost converter circuit 5306, and an output 5304 at a second voltage level. The buck-boost converter circuits 5200, 5300 are configured for different input voltage ranges. As shown in FIG. 8, the buck-boost converter circuit 5300 is loaded by R2 and R1 such that there is current (I) through the output 5304 terminal. For example, the buck-boost converter circuits 5200, 5300 are part of surgical stapling instrument 5 shown in FIGS. 1 and 3.

FIG. 7 is a graphical illustration of operational modes of an example DC/DC converter type buck-boost converter circuit such as the unloaded buck-boost converter circuit 5200 shown in FIG. 6, in accordance with the present disclosure. Three modes 5212, 5214, 5216 are depicted in a graph 5210, which represents EFFICIENCY (%) (along the vertical axis) as a function of INPUT VOLTAGE (V) (along the horizontal axis) applied to the buck-boost converter circuit 5200. For example, the boost mode 5212 occurs when the input voltage is approximately 400 mV below the output voltage. The buck mode 5216 occurs when the input voltage is approximately 800 mV above the output voltage. In the 4-Switch mode 5214, the input voltage is between the voltage for the boost mode 5212 and the buck mode 5216. The different modes are based on the input voltage level and the desired output voltage level (second voltage level). The modes vary in efficiency, with buck mode 5216 having the highest efficiency.

By way of example and with reference back to FIGS. 4 and 5, the control circuits 5012, 5112 determine the output voltage to the motor 5026, 5126. The operating modes of the converter circuits 5006a, 5006b, 5106a, 5106b, 5106c are based on the output voltage. The control circuits 5012, 5112 store the operating efficiencies of each operating mode for each of the converter circuits 5006a, 5006b, 5106a, 5106b, 5106c. The control circuit 5012, 5112 chooses the buck-boost converter circuit based on the output voltage and the operating efficiency of the operating mode of the converter circuit for that output voltage. Once the control circuit 5012, 5112 selects the operating mode for a converter circuit, the control circuit 5012, 5112 communicates to the control logic and register circuit 5008, 5108 via the SPI 5024, 5124 of the power management circuit 5010, 5110 to select the desired power supply output for the motor 5026, 5126 and other circuits as described above. For example, the control circuit 5012 may select one of the converter circuits 5006a, 5006b for connection to the motor 5026. For example, the control circuit 5112 may select one of the converter circuits 5106a, 5106b, 5106c to be connected to the motor 5126.

FIG. 9 is a graphical illustration of power loss and efficiency at different load currents for a buck-boost DC/DC converter type buck-boost converter circuit such as the loaded buck-boost converter circuit 5300 shown in FIG. 8, in accordance with the present disclosure. The graph 5220 illustrates EFFICIENCY (%) (along the left vertical axis) as a function of LOAD CURRENT (mA) (along the horizontal axis) and POWER LOSS (mW) (along the right vertical axis) as a function of LOAD CURRENT (mA). As shown, both efficiency 5222 and power loss 5224 increase as the load current increases.

By way of example and with reference back to FIGS. 4 and 5, the control circuits 5012, 5112 measure the load current to determine the efficiency and power loss of the converter circuits 5006a, 5006b, 5106a, 5106b, 5106c. The control circuits 5012, 5112 store the profile of power loss and efficiency for different load currents for each converter circuit 5006a, 5006b, 5106a, 5106b, 5106c. The control circuits 5012, 5112 can store the efficiency and power loss for different load currents for each converter circuit 5006a, 5006b, 5106a, 5106b, 5106c. The control circuits 5012, 5112 determine the power source based on at least one of the power loss, efficiency, or load current. By way of example and with reference back to FIG. 7, the control circuits 5012, 5112 can determine the efficiency based on the stored operating efficiencies of each of the modes for each converter circuits 5006a, 5006b, 5106a, 5106b, 5106c.

FIG. 10 illustrates a basic topology of a buck converter circuit 5400 in accordance with this disclosure. The buck converter circuit 5400 can be any one of the converter circuits (5006a, 5006b, 5106a, 5106b, 5106c) of FIGS. 4 and 5 The buck converter circuit 5400 has both inductor conduction losses and transistor conduction losses. The buck converter circuit 5400 comprises a first transistor 5402 (Q1) and a second transistor 5404 (Q2) coupled between an input 5406 and an output 5408 of the buck converter circuit 5400. A first current (shown in FIG. 12) flows through the first transistor 5402. A second current (shown in FIG. 13) flows through the second transistor 5404.

The input 5406 is coupled to a power source, such as the battery for the surgical instrument. The output 5408 can be coupled to the motor. The control circuit 5012, 5112 (shown in FIGS. 4 and 5) controls the level of the output voltage of the buck converter circuit 5400. The control circuit 5012, 5112 controls the transistors 5402 (Q1), 5404 (Q2) to change the output current I_{OUT} of the buck converter circuit 5400. The control circuit 5012, 5112 applies control signals to the inputs 5402a, 5402b of the transistors 5402 (Q1), 5404 (Q2) to control the current I_{Q1} output by the first transistor 5402 (Q1) and the current I_{Q2} output by the second transistor 5404 (Q2) to control the output current I_{OUT} of the buck converter circuit 5400.

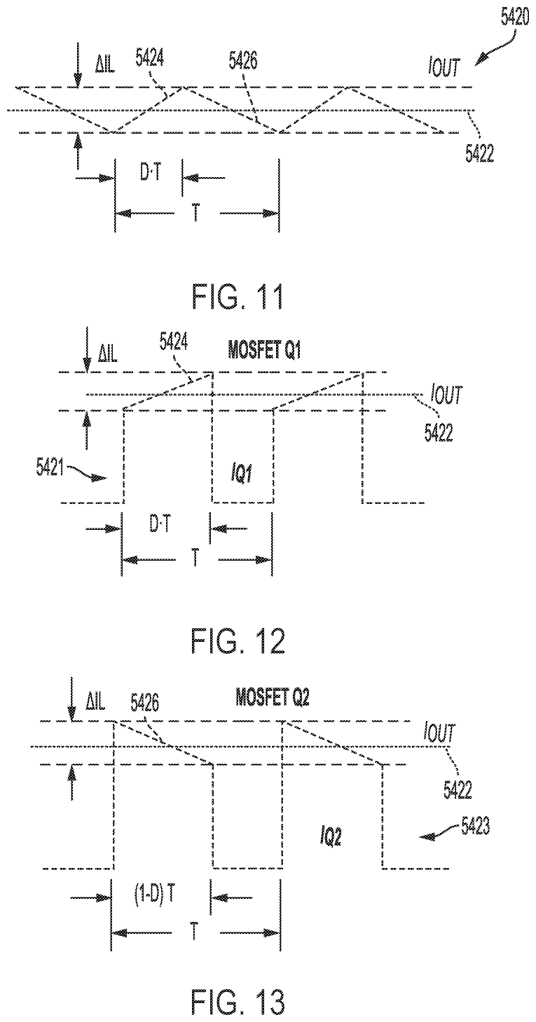

FIG. 11 illustrates the output current of the buck converter circuit 5400 I_{OUT} shown in FIG. 10. FIG. 12 illustrates the current I_{Q1} flowing through the first transistor 5402 (Q1) in FIG. 10. FIG. 13 illustrates the current I_{Q2} flowing through the second transistor 5404 (Q2) in FIG. 10. The current I_{Q1} flowing through the first transistor 5402 (Q1) and the current I_{Q2} flowing through the second transistor 5404 (Q2) produce the ripple current 5420 flowing through the inductor 5410 (L). The output current I_{OUT} 5422 resulting from the inductor L is applied to the motor (e.g., motor 5026, 5126 shown in FIGS. 4 and 5) through the output 5408.

The control circuit 5012, 5112 (shown in FIGS. 4 and 5) switches the first transistor 5402 (Q1) and the second transistor 5404 (Q2) on and off. For example, the control circuit 5012, 5112 turns on the first transistor 5402 (Q1) and turns off the second transistor 5404 (Q2). In the on state, while the first transistor 5402 (Q1) switch is conducting, the current through the inductor 5410 begins increasing linearly, as shown by the slope 5424 (FIGS. 11 and 12) of the first transistor 5402 (Q1) current 5421 (I_{Q1}). At the end of the duty cycle, the control circuit 5012, 5112 switches the first transistor 5402 (Q1) off.

The control circuit 5012, 5112 (shown in FIGS. 4 and 5) switches the second transistor 5404 (Q2) on. At this time, the inductor 5410 (L) continues conducting current and the second transistor 5404 (Q2) conducts current 5423 (I_{Q2}) (FIG. 13). The current through the inductor 5410 (L) begins decreasing linearly as shown by the slope 5426 (FIG. 13). The buck converter circuit waveforms representations of the ripple current 5420, 5421, 5423 shown in FIGS. 11-13 depict this switching action. The ripple current 5420 through the inductor 5410 (L) is shown in FIG. 11. As described above, I_{OUT} is the output current 5422 (FIGS. 11-13) through the output 5408 of the buck converter circuit 5400. For example, the output current 5422 is supplied to the motor of the surgical stapling instrument 5 shown in FIGS. 1 and 3.

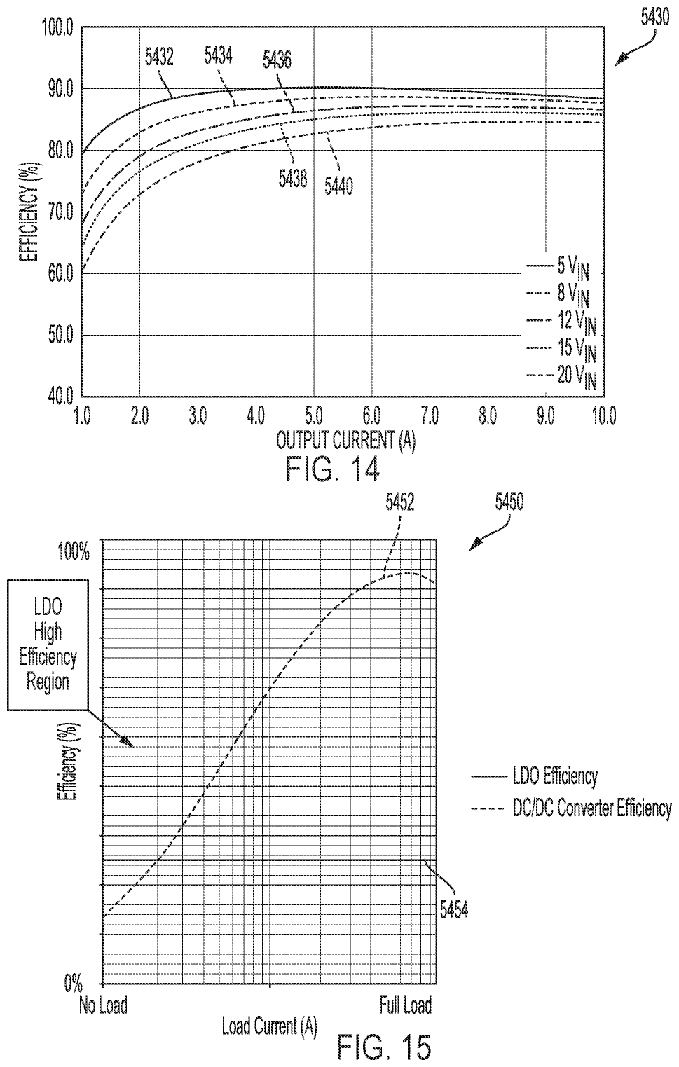

FIG. 14 is a graph 5430 of EFFICIENCY (%) (along the vertical axis) as a function of OUTPUT CURRENT (A) (along the horizontal axis) over a range of output currents of the buck converter circuit 5400 shown in FIG. 10 at discrete voltage values. The graph 5430 shows efficiency of the buck converter circuit 5400 for different input voltage values 5432 (5V), 5434 (8V), 5436 (12V), 5438 (15V), and 5440 (20V) over a range of output current I_{OUT} (1.0 A-10.0 A). As shown by the various plots, the efficiency is greater at lower input voltages. The control circuit 5012, 5112 (shown in FIGS. 4 and 5) stores similar efficiency profiles.

FIG. 15 is a graph 5450 of Efficiency (%) (along the vertical axis) as a function of Load Current (A) over a range of load currents for a LDO regulator, such as the LDO regulator 5004, 5104 (FIGS. 4 and 5) and a DC/DC converter type buck-boost converter circuits 5200, 5300 (FIGS. 6 and 8). As shown in FIG. 15, the efficiency 5452 of the DC/DC converter type buck-boost converter circuits 5200, 5300 increases with load current, whereas the efficiency 5454 of the LDO regulator 5004, 5104 does not change with load current. The efficiency of the LDO regulator 5004, 5104 is greater than the efficiency of the DC/DC converter type buck-boost converter circuits 5200, 5300 at low load currents. The control circuit 5012, 5112 (FIGS. 4 and 5) stores the efficiency of the LDO regulator 5004, 5104 (FIGS. 4 and 5), the converter circuits 5006a, 5006b, 5106a, 5106b (FIGS. 4 and 5), and the DC/DC converter type buck-boost converter circuits 5200, 5300 (FIGS. 6 and 8). The control circuit 5012, 5112 employs the stored efficiencies to determine which power supply is more efficient based on the present load condition of the motor 5026, 5126 (FIGS. 4 and 5). Accordingly, the control circuit 5012, 5112 may employ a current level operating range switching technique to optimize the power supply for its expected operating range to increase the efficiency. Accordingly, depending on the range, a DC/DC converter circuit may be more appropriate than an LDO regulator. Switching losses that occur in the circuits 5000, 5100 (FIGS. 4 and 5) of the surgical stapling instrument 5 (FIGS. 1-3) need to be minimized. To minimize the switching losses of the circuits 5000, 5100 of the surgical stapling instrument 5, the control circuits 5012, 5112 control the turn on/turn off times and the rise/fall times of the transistors of the converter circuits 5006a, 5006b, 5106a, 5106b, 5106c to minimize the switching circuitry losses. The control circuit 5012, 5112 controls the switching frequency to be above the human audible range.

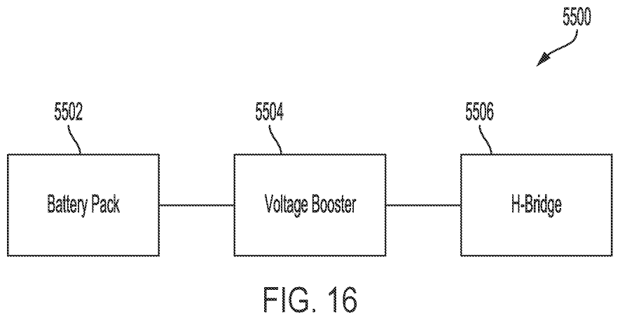

FIG. 16 illustrates a surgical system 5500, in accordance with the present disclosure. The surgical system 5500 is a block diagram of the surgical stapling instrument 5 shown in FIGS. 1-3. The surgical system 5500 comprises a battery pack 5502, an H-bridge 5506, and a voltage boost converter circuit 5504 coupled between the battery pack 5502 and the H-bridge 5506. Inserting the voltage boost converter circuit 5504 between the battery pack 5502 and the H-bridge 5506 may allow the surgical system 5500 to operate at lower voltages, however, the switching efficiency creates system losses. The surgical system 5500 can provide additional voltage to the voltage supplied by the battery pack 5502 to increase the voltage applied to the motor 5026, 5126 (FIGS. 4 and 5). The control circuit 5012, 5112 (FIGS. 4 and 5) can determine if the voltage boost converter circuit 5504 provides additional voltage to the H-bridge 5506. The control circuit 5012, 5112 determines which voltage boost converter circuit can provide additional voltage to the motor 5006, 5126 (FIGS. 4 and 5). Different types of voltage boost converter circuits 5504 are disclosed herein.

FIG. 17 illustrates an example implementation of a voltage boost converter circuit 5510, in accordance with the present disclosure. The voltage boost converter circuit 5510 can be any one of the converter circuits (5006a, 5006b, 5106a, 5106b, 5106c) of FIGS. 4 and 5. The voltage boost converter circuit 5510 produces an output voltage 5516 (V_{OUT}) based on an input voltage 5514 (V_{IN}) that is selectively coupleable to the motor 5026, 5126 (FIGS. 4 and 5). Increasing the capacitance of the output capacitor 5518 (C_{O}) reduces the effort required by the voltage boost converter circuit 5510 to maintain the output voltage 5516 (V_{OUT}) under current loaded conditions. The voltage boost converter circuit 5510 can regulate the output voltage 5516 (V_{OUT}) using current-mode, pulse-width modulation (PWM) control, for example. The voltage boost converter circuit 5510 receives the input voltage 5514 (V_{IN}) and the boost regulator 5512 outputs the output voltage 5516 (V_{OUT}). The control circuit 5012, 5112 (FIGS. 4 and 5) controls the boost regulator 5512 to set the output voltage 5516 (V_{OUT}).

The voltage boost converter circuit 5510 can maintain the output voltage 5516 (V_{OUT}) under high current loads from the motor 5026, 5126 (FIGS. 4 and 5). The control circuit 5012, 5112 (FIGS. 4 and 5) determines motor 5026, 5126 load is greater than a pre-determined threshold and couples the voltage boost converter circuit 5510 output to the motor 5026, 5126. This provides additional voltage to the motor 5026, 5126 to overcome any voltage sag that occurs due to load conditions.

The voltage boost converter circuit 5510 provides several advantages. For example, under heavy loading conditions of the battery pack, such as for example the battery pack 5502 shown in FIG. 16, the battery pack voltage sags considerably. Under considerable voltage sag, the electrical system of the surgical stapling instrument 5 (FIGS. 1-3) can go into a brown out condition. The voltage boost converter circuit 5510 can additionally boost the system voltage to maintain the desired voltage to the motor 5026, 5126 (FIGS. 4 and 5). The voltage boost converter circuit 5510 can be very efficient under high loading conditions.

For example, the electrical system of the surgical stapling instrument 5 (FIGS. 1-3) can incorporate an additional boost regulator that is isolated from the motor 5026, 5126 (FIGS. 4 and 5) subsystem only. This prevents sagging of the voltage applied to the motor. By minimizing the voltage sag, the circuit 5000, 5100 (FIGS. 4 and 5) of the surgical stapling instrument 5 can supply more power to the motor 5026, 5126 based on the desired motor operating curve. The voltage boost converter circuit 5510 can boost the nominal 12V system voltage to a higher voltage level.

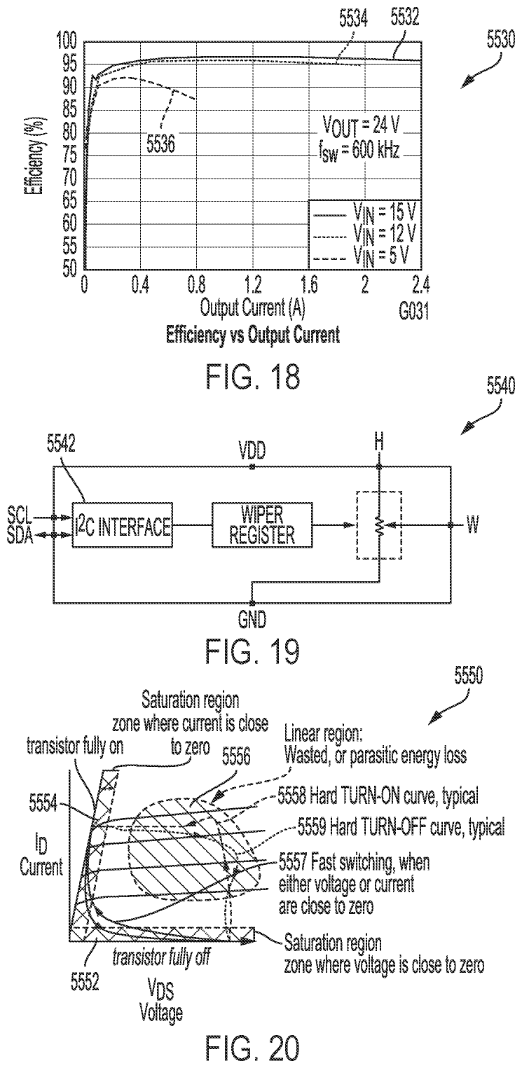

FIG. 18 is a graph 5530 illustrating efficiency (%) as a function of output current (A) for a DC/DC boost regulator. The graph 5530 shows the efficiency of a DC/DC boost regulator at different input voltage (V_{OUT}) levels over a range of output currents. The 5V input voltage 5536 is the least efficient. The 15V input voltage 5532 is more efficient than 12V input voltage 5534.

FIG. 19 is a variable potentiometer 5540, in accordance with the present disclosure. The variable potentiometer 5540 is controlled by the input received at the serial interface circuit 5542 (e.g., an I²C interface circuit) from the control circuit 5012, 5112 (FIGS. 4 and 5). The received input controls wiper registers 5544 to control the potentiometer 5546 and change the resistance of the potentiometer 5546.

In accordance with the present disclosure, the variable resistor 5540 can be adjustable in “real” time based on the system performance by the control circuit 5012, 5112 (FIGS. 4 and 5). The variable potentiometer 5540 can be used as the first resistor 5520 (R_{SH}) and/or the second resistor 5522 (R_{SL}) of the voltage boost converter circuit 5510 (FIG. 17). The resistance of the first resistor 5520 (R_{SH}) and/or the second resistor 5522 (R_{SL}) are then set by the control circuit 5012, 5112 to vary the output voltage 5516 (V_{OUT}) of the voltage boost converter circuit 5510 and thus set the speed of the motor 5026, 5126 (FIGS. 4 and 5).

In accordance with the present disclosure, motor load feedback can be a measured parameter and the resistance values of at least one of the first resistor 5520 (R_{SH}) and the second resistor 5522 (R_{SL}) (FIG. 17) can be adjusted based on at least the load on the motor 5026, 5126 (FIGS. 4 and 5). The potentiometer 5546 can be a dynamic active resistor. The dynamic resistor changes the resistance electronically.

In accordance with the present disclosure, the control circuit 5012, 5112 (FIGS. 4 and 5) can control the converter circuits to dynamically change speed of the motor. Further, the control circuit can switch between static boost circuits to change the speed of the motor. The converter circuits 5006a, 5006b, 5106a, 5106b, 5106c (FIGS. 4 and 5), and specific implementations of such circuits described above, can be controlled by the control circuit 5012, 5112 to lower the voltage applied to the motor 5026, 5126 (FIGS. 4 and 5).

FIG. 20 is a graph 5550 of drain current (ID) as a function of drain-to-source voltage (VDs) for switching a field effect transistor, in accordance with the present disclosure. For MOSFETs, IGBTs, and other transistors, moving these components out of their linear zones as quickly as possible saves energy because the time it takes for the transistor to turn on is all wasted or is a parasitic loss. The graph 5550 illustrates a first saturation region 5552 where the voltage is close to zero. The transistor is off in the first saturation region 5552. The graph 5550 also illustrates a second saturation region 5554 where the current is close to zero. The transistor is on in the second saturation region 5554. The graph 5550 also illustrates a linear region 5556.

Driving the transistor hard out of the linear region and into the saturation region quickly saves energy. In addition, switching between the linear and saturation states can be done as fast as possible to minimize losses. The graph 5550 illustrates the hard turn on curve 5558 and the hard turn off curve 5559. The control circuit 5012, 5112 (FIGS. 4 and 5) controls the speed at which the transistor is switched between the linear and saturation states to minimize losses.

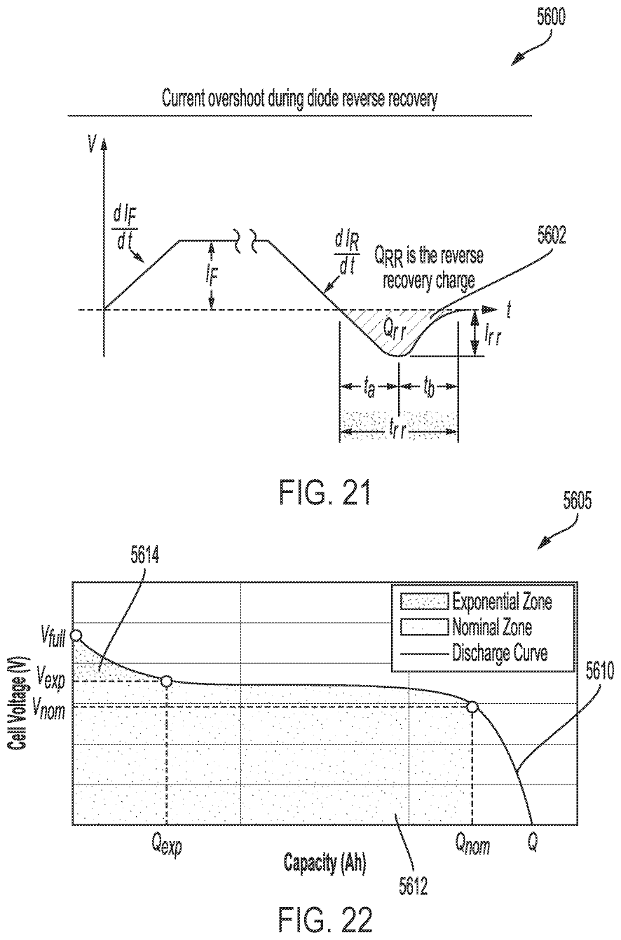

FIG. 21 is a graphical depiction of leakage current losses, in accordance with the present disclosure. The graph 5600 depicts current overshoot during diode reverse recovery period. The graph 5600 shows voltage (V) as a function of time (t) where Q_{RR} is the reverse recovery charge 5602 over period tᵣᵣ. For example, operating an integrated circuit near or above the maximum operating temperature and/or voltage increases the leakage current. The control circuit 5012, 5112 (FIGS. 4 and 5) can control the operation of an integrated circuit to lower the operating temperature based on a measured operating temperature. A temperature sensor can measure the operating temperature of the integrated circuit.

In accordance with the present disclosure, the use of fast recovery diodes may improve switching losses, but increases conduction losses. To reduce losses, the components of the overall electrical circuitry that are not in use should thus be put in a sleep state or turned off. These small currents can add up to a substantial “parasitic” current draw of the power source. The graph 5600 of reverse recovery illustrates that the reverse recovery charge 5602 is based on the current and the required recovery time.

FIG. 22 is a graph 5605 of a battery discharge curve 5610, in accordance with the present disclosure. The graph 5605 depicts the Cell Voltage (V) as a function of Capacity (Ah). The battery discharge curve 5610 illustrates the voltage drop of a battery over the amp hours that the battery is used. The exponential zone 5614 represents the exponential voltage drop of the battery from full voltage to exponential voltage level. The nominal zone 5612 represents the area where the voltage drops from the exponential voltage to the nominal voltage.

FIG. 22 will now be described together with FIGS. 1-5 and 16. In accordance with the present disclosure, to maintain peak power to the motor system, maintaining a maximized potential voltage is a key parameter. As the cell capacity of a battery decreases, the cell voltage also trends downward. Thus, the power applied to the motor is affected by the amp-hours (Ah) that the battery has been used for. Motor power is the product of voltage and current. Battery voltage reduction has a direct impact on the power the system can supply to the motor.

For example, the surgical stapling instrument 5 (FIGS. 1-3) can employ Lithium batteries as its power source. These cells have a capacity of approximately 1500 mAh with a nominal voltage of 3V per cell. The battery pack, such as the battery pack 5502 (FIG. 16), is able to deliver all the required power for up to 12 hours. The current set up requires approximately 50 mA to run. At 12 hours of idle, the system has consumed 50 mA×12 hours=600 mAh. This equates to consuming 40% of the battery capacity in an idle or non-productive manner.

With reference to FIGS. 4 and 5, to minimize these losses, reductions in parasitic losses and power management techniques are required. As previously discussed, to reduce losses, components of the overall electrical circuitry that are not in use should thus be put in a sleep state or turned off. These small currents can add up to a substantial “parasitic” draw of the power source. The circuit 5000, 5100 may comprise a power management circuit 5010, 5110. The circuit 5000, 5100 controls the power to the non-therapeutic tissue sensing circuits.

Further, in accordance with the present disclosure, the power management circuit 5010, 5110 can monitor a sensor or circuit element to determine when to turn on and off the surgical stapling instrument 5 (FIGS. 1-3) and the circuit elements to be energized. The sensor can be at least one of a gyroscope, accelerometer, thermal sensor, pressure sensor, or any other type of sensor to determine activity of the surgical stapling instrument 5 or a timer for a surgical stapling instrument 5 timeout after a pre-determined amount of time without activity. Activity of the surgical stapling instrument 5 includes picking up the surgical stapling instrument 5. The activity is determined by the gyroscope or accelerometer. A user's hand on the surgical stapling instrument 5 can be detected by a pressure or temperature sensor. The orientation of the surgical stapling instrument 5 can be determined by the gyroscope. The motion of the surgical stapling instrument 5 can be determined by the accelerometer. Accordingly, any indication that the surgical stapling instrument 5 is going to be used by a user can be detected by a suitable sensor.

In accordance with the present disclosure, the power management circuit 5010, 5110, under control of the control circuit 5012, 5112, may engage or disengage unused electrical components. For example, the power management circuit is to engage circuit components when the device determines an activity has occurred. The power management circuit is to couple the electrical components of the circuit to a power source to engage the surgical stapling instrument 5. The power management circuit 5010, 5110 disengages when the surgical stapling instrument 5 determines that there has been a lack of activity for a pre-determined amount of time or that the activity has ended. Disengaging may be severing a connection between the unused electrical components and the power source.

In accordance with the present disclosure, the end of activity can be determined by the orientation of the surgical stapling instrument 5 (FIGS. 1-3), the lack of movement of the surgical stapling instrument 5, the lack of a user's hand on the surgical stapling instrument 5, or any indication that the user is done employing the surgical stapling instrument 5. The power management circuit 5010, 5110 is to disengage circuit components from the battery based on the determination that the activity has ended. Disengaging includes disconnecting circuit components from the battery to prevent loss when not in use.

In accordance with the present disclosure, the power management circuit 5010, 5110 can electrically disengage, severs, or disconnect circuit components that are not is use while operating the surgical stapling instrument 5 (FIGS. 1-3). The power management circuit 5010, 5110 may disengage circuit components based on the voltage level of the power source and the load on the motor 5026, 5126.

Further, in accordance with the present disclosure, the power management circuit 5010, 5110 can interact with external mechanisms or features to determine activity of the surgical stapling instrument 5 (FIGS. 1-3). The power management circuit 5010, 5110 engages circuit components based interactions with the external mechanisms. For example, the power management circuit 5010, 5110 can interact with a trocar. The power management circuit 5010, 5110 engages circuit components, which activate the surgical stapling instrument 5. For example, insertion of the surgical stapling instrument 5 into a trocar turns the surgical stapling instrument 5 on and removal of the surgical stapling instrument 5 from the trocar turns the surgical stapling instrument 5 off or to an idle state.

In accordance with the present disclosure, the external feature of the surgical stapling instrument 5 (FIGS. 1-3) can be a staple cartridge 22 (FIGS. 1 and 2). The power management circuit 5010, 5110 determines the insertion of the staple cartridge 22 into a channel 21 (FIG. 1) of the surgical stapling instrument 5. The power management circuit 5010, 5110 engages circuit components based on the insertion of the staple cartridge 22 into the channel 21. The power management circuit 5010, 5110 determines the staple cartridge 22 is empty and disengages circuit components based on the determination.

Further, in accordance with the present disclosure, the external feature can be a device located in the operating room. The power management circuit 5010, 5110 determines that the surgical stapling instrument 5 (FIGS. 1-3) is located in the operating room or that a second device is located in the operating room. The power management circuit 5010, 5110 engages circuit components based on the determination that the surgical stapling instrument 5 is located in the operating room or based on the determination that a second device is located in the operating room. For example, the second device indicates that a surgical procedure is to start. The second device may be another surgical stapling instrument similar to the surgical stapling instrument 5.

In accordance with the present disclosure, the power management circuit 5010, 5110 can interact with devices externally connected through a wireless connection. The externally connected devices inform the power management circuit 5010, 5110 as to its status during the surgical procedure. Additionally, the power management circuit 5010, 5110 can receive signals from an externally connected device coupled to a surgical hub or surgical energy generator hub (e.g., ultrasonic, monopolar or bipolar RF, or any combination of ultrasonic, monopolar or bipolar RF energy). Some of the received signals indicate that there will be a delay before the externally connected device will be required again or that the externally connected device service in the procedure is complete. The externally connected device can be partially or completely turned off based on the signals. Alternatively, the externally connected devices can be partially or completely turned on by the signals. Engaging circuit components may include turning on the externally connected device or the surgical stapling instrument 5 (FIGS. 1-3) and disengaging circuit components.

Further, in accordance with the present disclosure, the power management circuit 5010, 5110 can control the user interface. The power management circuit 5010, 5110 adjusts the brightness or contrast of a display portion of the surgical stapling instrument 5. For example, during a firing stage of the surgical stapling instrument 5, the power management circuit 5010, 5110, controls the display. In accordance with the present disclosure, a user may not look at the display until the firing stage is complete. Accordingly, during the firing stage the display is turned off and the display is turned on after the firing stage.

Further, in accordance with the present disclosure, the power management circuit 5010, 5110 may disengage circuit components while the surgical stapling instrument 5 (FIGS. 1-3) is in a storage or shipping package. The power management circuit 5010, 5110 determines that the surgical stapling instrument 5 has been removed from the storage or shipping package based on input received from various sensors. The power management circuit 5010, 5110 maintains the device in idle or off state until after the storage or shipping package is removed and the surgical stapling instrument 5 is initialized. The sensor can be a pressure sensor to detect a broken vacuum seal in the storage or shipping package. The pressure sensor also can sense that a user is holding the surgical stapling instrument 5. The sensor can be a temperature sensor to detect that the user is holding the device. A switch on the surgical stapling instrument 5 can activate the surgical stapling instrument 5 for the first time and can initialize the surgical stapling instrument 5. Any other suitable sensor or method can be employed to sense removal of the surgical stapling instrument 5 from the storage or shipping package.

Additionally, in accordance with the present disclosure, the power management circuit 5010, 5110 can also control the power management of the communication systems. The power management circuit 5010, 5110 may control the data rate of the communication system. The control circuit 100 (FIG. 3), 5012 (FIG. 4), 5112 (FIG. 5) adjusts the data rate based on at least the power level of the battery. The control circuit, 100, 5012, 5112 adjusts the data rate based on at least the hardware connected to the surgical stapling instrument 5 (FIGS. 1-3).

Additionally, in accordance with the present disclosure, the power management circuit 5010, 5110 may disable the communication systems based on a lack of detecting associated hardware in the operating room. The power management circuit 5010, 5110 determines a lack of hardware in the operation room based on: (1) no detection of devices within a received signal strength indication link range, (2) no devices acting as a preparing advertising beacon, and (3) a lack of audio-based communication, infrared IR based communication, and RFID signals.

Further, in accordance with the present disclosure, the power management circuit 5050, 5110 may not be part of the control circuit 100 (FIG. 3), 5012 (FIG. 4), 5112 (FIG. 5). The power management circuit 5010, 5110 can selectively engage and disengage circuit components based on at least one of the voltage level of the power source, the state of the surgical stapling instrument 5 (FIGS. 1-3), the desired power level to the motor 5026, 5126 (FIGS. 4-5), and the circuit components to be used.

In accordance with the present disclosure, the power management circuit 5010, 5110 can charge a super capacitor. When additional power is required to service the electrical system, the super capacitor can deliver an additional pulse of energy into the system. The energy can be a burst mode of short duration.

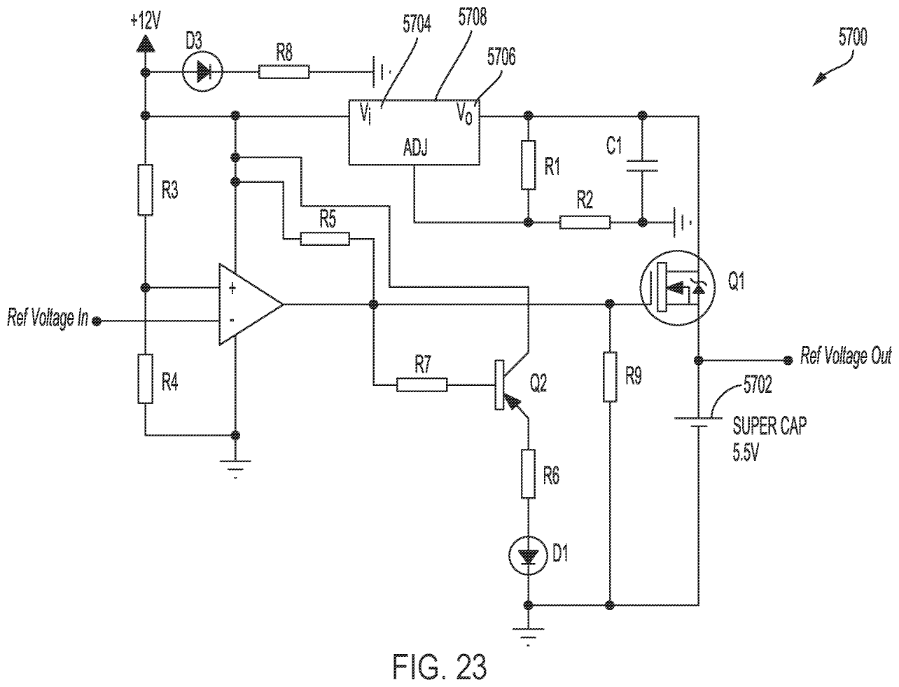

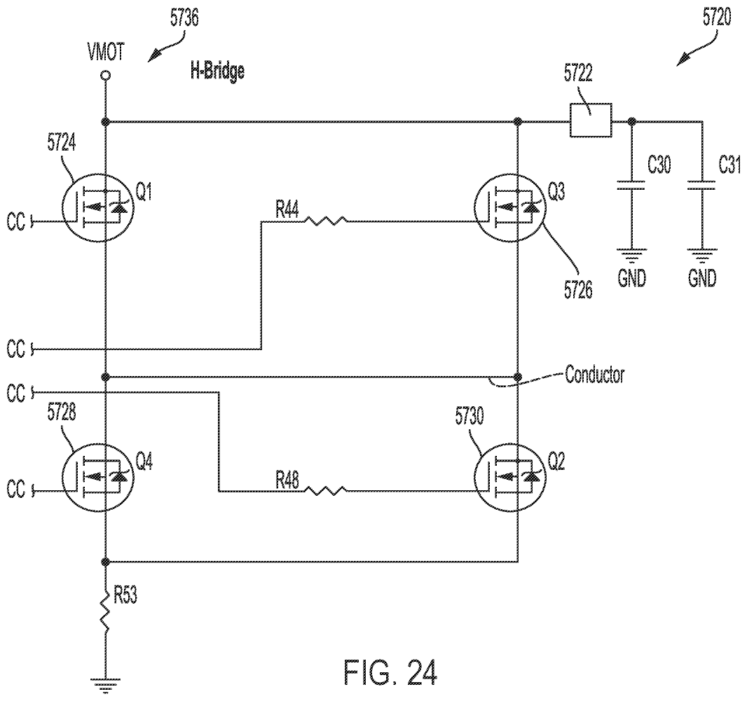

FIG. 23 is a super capacitor charging circuit 5700, according to this disclosure. The super capacitor charging circuit 5700 comprises an adjustable regulator 5708 to charge a super capacitor 5702. The adjustable regulator 5708 comprises an input 5704 to receive a first voltage level and an output 5706 to output a second voltage level to charge the super capacitor 5702. Super capacitors can be integrated into the H-Bridge motor drive circuit 5720 (shown in FIG. 24).

For example, the control circuit 100 (FIG. 3), 5012 (FIG. 4), 5112 (FIG. 5) of the surgical stapling instrument 5 (FIGS. 1-3) detects thick dense tissue within the anvil 24 and the staple cartridge 22 of the end effector 20 (FIGS. 1-3) during a drive cycle of the surgical stapling instrument 5. The control circuit 100 can detect the thick dense tissue by monitoring the voltage, current, and the speed of the knife coupled to the firing beam 60 (FIG. 1). If the monitored parameters begin to fall below a minimum threshold for a successful drive cycle, the control circuit 100, 5012, 5112 triggers the inclusion of a charged super capacitor 5702 into the control circuit 100, 5012, 5112. The stored energy in the super capacitor 5702 is added to the drive system power to increase the output power level above the standard levels. The increased power provided by switching in the super capacitor 5702 provides additional energy to the drive system to complete the drive cycle.

FIG. 24 illustrates a motor drive circuit 5720 with boost circuit connections, in accordance with the present disclosure. As illustrated in FIG. 24, the motor drive circuit 5720 can be an H-bridge. Other drive circuit implementations are contemplated to be within the scope of this disclosure. The motor drive circuit 5720 controls the function of the motor, such as for example, the motor 5026, 5126 (FIGS. 4 and 5). By turning on a first transistor 5724 (Q1) and a second transistor 5730 (Q2) and turning off a third transistor 5726 (Q3) and a fourth transistor 5728 (Q4), the motor is energized in a forward operation. The positive motor lead is connected between the first transistor 5724 (Q1) and the fourth transistor 5728 (Q4). The negative motor lead is connected between the second transistor 5730 (Q2) and the third transistor 5726 (Q3). By turning on the third transistor 5726 (Q3) and the fourth transistor 5728 (Q4) and turning off the first transistor 5724 (Q1) and the second transistor 5730 (Q2), the motor is energized in a backward operation. The voltage applied to the motor is supplied from a power source 5736.

In accordance with the present disclosure, the supercapacitor circuit (FIG. 23) may be coupled to the motor drive circuit 5720 by a switch 5722. The switch 5722 couples the motor drive circuit 5720 and the super capacitor charging circuit 5700. When the switch 5722 is closed, the super capacitor charging circuit 5700 provides additional power to the motor. When the switch 5722 is open, the motor is driven by the power source 5736.

The switch 5722 is controlled by the control circuit 100, 5012, 5112 (FIGS. 3-5). The control circuit 100, 5012, 5112 monitors any voltage sag of the power source 5736. The control circuit 100, 5012, 5112 determines that the voltage sag is greater than a pre-determined threshold and based on the determination the control circuit 100, 5012, 5112 closes the switch 5722 to supply additional power from the super capacitor 5702 (FIG. 23).

In accordance with the present disclosure, the power boost can also prevent a brownout condition of the electronics for lack of sufficient power to the electronics.

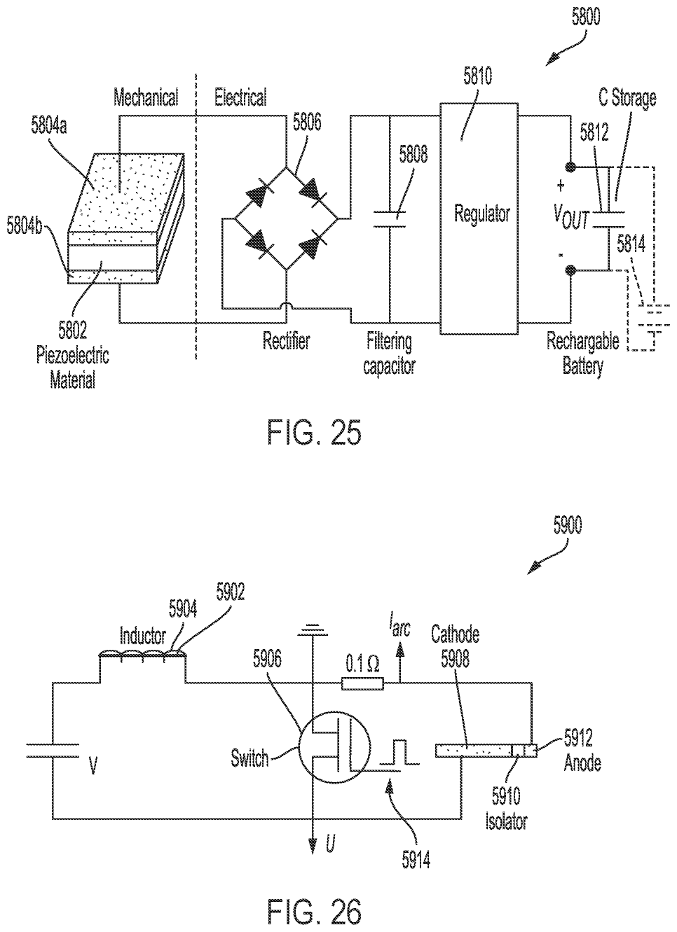

FIG. 25 is a vibration circuit 5800 to harvest energy from vibrations of the surgical stapling instrument 5 (FIGS. 1-3), in accordance with the present disclosure. The vibration circuit 5800 generates and stores power based on the vibration of the surgical stapling instrument 5. The vibration circuit 5800 can harvest energy from the inherent vibrations experienced by the surgical stapling instrument 5 and can store the energy for future uses. Energy harvested from the surgical stapling instrument 5 vibrations also may be used to slowly charge an energy storage system such as a capacitor, rechargeable battery, and the like.

In accordance with the present disclosure, the vibration circuit 5800 may comprise a piezoelectric material 5802 coupled between a first electrode 5804a and a second electrode 5804b. Under mechanical vibration the piezoelectric material 5802 generates an alternating voltage across the first 5804a and second electrodes 5804b. The vibration circuit 5800 also comprises a rectifier 5806 to convert the alternating voltage into a DC output voltage. The vibration circuit 5800 also comprises a filtering capacitor 5808. The vibration circuit 5800 also comprises a regulator 5810 to maintain a fixed output voltage irrespective of input voltage or load conditions. The voltage at the output of the regulator 5810 can be used to charge a storage capacitor 5812 (C_{storage}) or charge a rechargeable battery 5814.

The collected power is stored and can be released if needed by the surgical stapling instrument 5 (FIGS. 1-3). For example, the control circuit 100, 5012, 5112 (FIGS. 3-5) determines that the desired voltage to the motor is less than the voltage received by the motor. The control circuit 100, 5012, 5112 discharges the stored power in the vibration circuit 5800 based on the determination.

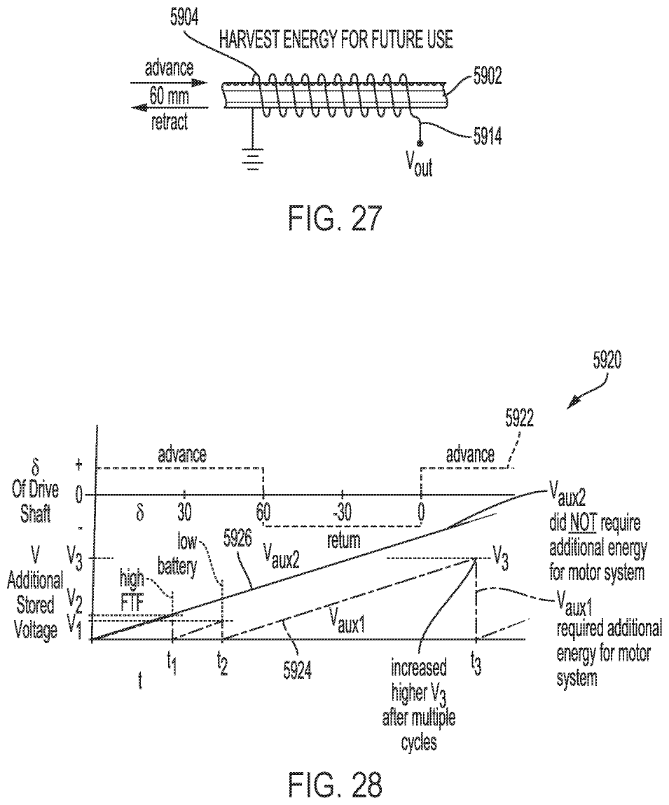

FIGS. 26 and 27 is a circuit 5900 to collect power from the movement of a drive shaft 5902 of the surgical stapling instrument (FIGS. 1-3) such as, for example, the drive shaft 40 (FIGS. 1 and 3), in accordance with the present disclosure.

FIG. 27 is a detailed view of the energy harvesting inductor 5904 showing a coil 5914 surrounding the drive shaft 5902. As illustrated in FIG. 26, the inductor 5904 can be formed by surrounding the drive shaft 5902 with a coil 5914. As the drive shaft 5902 moves back and forth with each firing cycle of the surgical stapling instrument 5, current is in the inductor 5904.

In accordance with the present disclosure, the circuit 5900 can store the energy in the inductor 5904 for future extraction. The collected energy is stored in the inductor 5904 and released if and when it is needed by the surgical stapling instrument 5 (FIGS. 1-3). For example, the control circuit 100, 5012, 5112 (FIGS. 3-5) determines that the desired voltage to the motor is less than the voltage received by the motor. Accordingly, the control circuit 100, 5012, 5112 discharges the stored energy in the circuit 5900 based on the determination.

FIG. 28 illustrates a graph 5920 of additional energy applied to the system from the circuit 5900, in accordance with the present disclosure. The graph 5920 illustrates the movement of the drive shaft 5902 shown in FIGS. 26-27. A first voltage profile 5924 illustrates the voltage when additional energy is required for the motor system. A second voltage profile 5926 illustrates the voltage when additional energy is not required for the motor. Additional energy is required when the output voltage is below the desired voltage to the motor.

In accordance with the present disclosure, the control circuit 100, 5012, 5112 (FIGS. 3-5) can separate a distinct battery sub-system from the rest of the circuit components in order to use the sub-system as an auxiliary pulsed power source.

In accordance with the present disclosure, a higher performance battery pack can be used during over stressed firing conditions. For example, a battery cell is at 3.0 volts, whereas other battery cells in the same size at 3.3 to 3.8 volts. The use of four battery cells with a 0.3 to 0.8 volt increase, increases the total voltage by 1.2 to 3.2 volts (10% to 26% increase). This battery system would have a longer life due to the increased initial voltage.

Examples of the apparatus and method according to the present disclosure are provided below in the following numbered clauses. An aspect of the apparatus or method may include any one or more than one, and any combination of, the numbered clauses described below.

Example 1—A surgical instrument comprising a motor (5026, 5126), a regulator (5004, 5104), a converter circuit (5006a, 5006b, 5106a, 5106b, 5106c), and a control circuit (100, 5012, 5112) coupled to the regulator and the converter circuit, wherein the control circuit is to: couple either the regulator or the converter circuit based on a pre-determined voltage or current load applied to the motor.

Example 2—The surgical instrument of Example 1, wherein the converter circuit is a boost converter circuit.

Example 3—The surgical instrument of Example 2, wherein the boost converter circuit is coupled based on the voltage load at the motor exceeding the pre-determined voltage load.

Example 4—The surgical instrument of any of Examples 2-3, wherein the control circuit is to: determine a voltage sag at the motor and couple the boost converter circuit to the motor.

Example 5—The surgical instrument of Example 1, wherein the converter circuit is a buck converter circuit.

Example 6—The surgical instrument of any of Example 5, wherein the buck converter circuit is coupled based on the current exceeding the pre-determined current load.

Example 7—The surgical instrument of any of Examples 1-6, wherein the regulator is a low dropout (LDO) regulator, and wherein the control circuit is to couple the motor to the LDO regulator based on the voltage load of the motor being below the pre-determined voltage load.

Example 8—The surgical instrument of any of Examples 1-7, further comprising at least one more converter circuit, wherein the converter circuit and the at least one more converter circuit are configured to power the motor for different load conditions.

Example 9—The surgical instrument of any of Examples 1-8, wherein the control circuit (5010, 5110) comprises a plurality of electrical components, wherein the control circuit is coupled to a power supply, and wherein the control circuit is to: determine unused electrical components of the plurality of electrical components and sever a connection between the unused electrical components and the power supply based on the electrical components being unused.

Example 10—The surgical instrument of any of Examples 1-9, wherein the control circuit comprises a power management circuit (100, 5012, 5112) to: determine a period elapsed since a previous activity; and determine that the period elapsed since the previous activity is greater than the pre-determined period; and turn off power to the surgical instrument based on the determination.

Example 11—The surgical instrument of any of Examples 1-10, wherein the control circuit is to: determine that the surgical instrument has been inserted in a surgical site; and turn on power to the surgical instrument based on a determination that the surgical instrument was inserted in the surgical site.

Example 12—The surgical instrument of any of Examples 7-11, wherein the control circuit is to: determine that the surgical instrument has been removed from a surgical site; and turn off power to the surgical instrument based on a determination that the surgical instrument was removed from the surgical site.

Example 13—The surgical instrument of any of Examples 7-12, wherein the control circuit is to: determine that the surgical instrument is separated from a packaging and turn on power to the surgical instrument.

Example 14—The surgical instrument of any of Examples 1-13, further comprising: a power supply, a power accumulator, and wherein the control circuit is coupled to the power supply and the power accumulator, wherein the control circuit is to: charge the power accumulator; determine that a parameter is below a threshold; and discharge power from the power accumulator to increase power output of the power supply based on the parameter being below the threshold.

Example 15—The surgical instrument of Example 14, wherein the power accumulator comprises at least one of: an inductor coiled around a drive bar to harvest energy from oscillatory movement of a drive bar and store the harvest energy in the inductor; or a boost circuit comprising a variable resistor or potentiometer to adjust an output voltage of the boost circuit.

Example 16—A surgical instrument comprising a motor, a regulator, a converter circuit, and a control circuit coupled to the regulator and the converter circuit. The control circuit is to: couple either the regulator, or the converter circuit based on a pre-determined load voltage or current applied to the motor.

Example 17—The surgical instrument of Example 16, wherein the converter circuit is a boost converter circuit.

Example 18—The surgical instrument of Example 17, wherein the boost converter circuit is coupled based on the voltage load at the motor exceeding the pre-determined voltage load.

Example 19—The surgical instrument of Example 17, wherein the control circuit is to determine a voltage sag at the motor and couple the boost converter circuit to the motor.

Example 20—The surgical instrument of any of Example 16, wherein the converter circuit is a buck converter circuit.

Example 21—The surgical instrument of Example 20, wherein the buck converter circuit is coupled based on the current exceeding the pre-determined current load.

Example 22—The surgical instrument of any of Examples 16-21, wherein the control circuit is to disconnect control electronics from a power source.

Example 23—The surgical instrument of any of Examples 16-22, wherein the regulator is a low dropout (LDO) regulator, and wherein the control circuit is to couple the motor to the LDO regulator based on the voltage load of the motor being below the pre-determined voltage load.

Example 24—The surgical instrument of any of Examples 16-23, further comprising at least one more converter circuit, wherein the converter circuit and the at least one more converter circuit are configured to power the motor for different load conditions.

Example 25—A surgical instrument comprising a power supply and a control circuit comprising a plurality of electrical components. The control circuit is coupled to the power supply. The control circuit is to determine unused electrical components of the plurality of electrical components and sever a connection between the unused electrical components and the power supply based on the electrical components being unused.

Example 26—The surgical instrument of Example 25, wherein the control circuit is to determine that a pre-determined period has elapsed since a previous activity and turn off power to the surgical instrument.

Example 27—The surgical instrument of Example 26, wherein the previous activity is based on a signal received by the control circuit from one of a gyroscope, accelerometer, thermal sensor, or pressure sensor.

Example 28—The surgical instrument of any of Examples 25-26, wherein the control circuit comprises a power management circuit to: determine a period elapsed since a previous activity; and determine that the period elapsed since the previous activity is greater than the pre-determined period; and turn off power to the surgical instrument.

Example 29—The surgical instrument of any of Examples 24-28, wherein the control circuit is to: determine that the surgical instrument has been inserted in a surgical site and turn on power to the surgical instrument based on a determination that the surgical instrument was inserted into the surgical site.

Example 30—The surgical instrument of any of Examples 24-29, wherein the control circuit is to: determine the surgical instrument has been removed from the surgical site; and turn off power to the surgical instrument based on a determination that the surgical instrument was removed from the surgical site.

Example 31—The surgical instrument of any of Examples 24-30, wherein the control circuit is to: determine that the surgical instrument is separated from packaging; and turn on power to the surgical instrument.

Example 32—A surgical instrument comprising: a power supply, a power accumulator, and a control circuit coupled to the power supply and the power accumulator, wherein the control circuit is to: charge the power accumulator, determine that a parameter falls below a threshold, and discharge power from the power accumulator to increase power output of the power supply based on the parameter being below the threshold.

Example 33—The surgical instrument of Example 32, wherein the power accumulator is a vibration system to collect power from vibrations of the surgical instrument, the vibration system comprising: a first electrode, a second electrode, and a piezoelectric material disposed between the first and second electrode.

Example 34—The surgical instrument of any of Examples 32-33, wherein the power accumulator comprises an inductor coiled around a drive bar to harvest energy from oscillatory movement of a drive bar and store the harvest energy in the inductor.

Example 35—The surgical instrument of any of Examples 32-34, wherein the power accumulator is a boost circuit comprising a variable resistor to adjust an output voltage of the boost circuit.

The foregoing detailed description has set forth various forms of the systems and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

Any of the software components or functions described in this application, may be implemented as software code to be executed by a processor using any suitable computer language such as, for example, Python, Java, C++ or Perl using, for example, conventional or object-oriented techniques. The software code may be stored as a series of instructions, or commands on a computer readable medium, such as RAM, ROM, a magnetic medium such as a hard-drive or a floppy disk, or an optical medium such as a CD-ROM. Any such computer readable medium may reside on or within a single computational apparatus and may be present on or within different computational apparatuses within a system or network.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

A network may include a packet switched network. The communication devices may be capable of communicating with each other using a selected packet switched network communications protocol. One example communications protocol may include an Ethernet communications protocol which may be capable of permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December, 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, the transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

Unless specifically stated otherwise as apparent from the foregoing disclosure, it is appreciated that, throughout the present disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more than one” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more than one” or “at least one” and indefinite articles such as “a” or “an” (e.g., “a” and/or “an” should typically be interpreted to mean “at least one” or “one or more than one”); the same holds true for the use of definite articles used to introduce claim recitations. The singular form of “a”, “an”, and “the” include the plural references unless the context clearly dictates otherwise.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material. None is admitted to be prior art.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

It is worthy to note that any reference numbers included in the appended claims are used to reference exemplary embodiments/elements described in the present disclosure. Accordingly, any such reference numbers are not meant to limit the scope of the subject matter recited in the appended claims.

1-15. (canceled)

16. A surgical instrument comprising:
a motor;
a regulator;
a converter circuit; and
a control circuit coupled to the regulator and the converter circuit, wherein the control circuit is to:
couple either the regulator or the converter circuit based on a pre-determined voltage or current load applied to the motor.

17. The surgical instrument of claim 16, wherein the converter circuit is a boost converter circuit.

18. The surgical instrument of claim 17, wherein the boost converter circuit is coupled based on a voltage load at the motor exceeding the pre-determined voltage load.

19. The surgical instrument of claim 17, wherein the control circuit is to:
determine a voltage sag at the motor; and
couple the boost converter circuit to the motor.

20. The surgical instrument of claim 16, wherein the converter circuit is a buck converter circuit.

21. The surgical instrument of claim 20, wherein the buck converter circuit is coupled based on a current exceeding the pre-determined current load.

22. The surgical instrument of claim 16, wherein the control circuit is to disconnect control electronics from a power source.

23. The surgical instrument of claim 16, wherein the regulator is a low dropout (LDO) regulator, and wherein the control circuit is to couple the motor to the LDO regulator based on the voltage load of the motor being below the pre-determined voltage load.

24. The surgical instrument of claim 16, further comprising at least one more converter circuit, wherein the converter circuit and the at least one more converter circuit are configured to power the motor for different load conditions.

25. A surgical instrument comprising:
a power supply; and
a control circuit comprising a plurality of electrical components, wherein the control circuit is coupled to the power supply, and wherein the control circuit is to:
determine unused electrical components of the plurality of electrical components; and
sever a connection between the unused electrical components and the power supply based on the electrical components being unused.

26. The surgical instrument of claim 25, wherein the control circuit is to:
determine that a pre-determined period has elapsed since a previous activity; and
turn off power to the surgical instrument.

27. The surgical instrument of claim 26, wherein the previous activity is based on a signal received by the control circuit from one of a gyroscope, accelerometer, thermal sensor, or pressure sensor.

28. The surgical instrument of claim 27, wherein the control circuit comprises a power management circuit to:
determine a period elapsed since a previous activity;
determine that the period elapsed since the previous activity is greater than the pre-determined period; and
turn off power to the surgical instrument based on the determination.

29. The surgical instrument of claim 25, wherein the control circuit is to:
determine that the surgical instrument has been inserted in a surgical site; and
turn on power to the surgical instrument based on a determination that the surgical instrument was inserted into the surgical site.

30. The surgical instrument of claim 25, wherein the control circuit is to:
determine that the surgical instrument has been removed from a surgical site; and
turn off power to the surgical instrument based on a determination that the surgical instrument was removed from the surgical site.

31. The surgical instrument of claim 25, wherein the control circuit is to:
determine the surgical instrument is separated from packaging; and
turn on power to the surgical instrument.

32. A surgical instrument comprising:
a power supply;
a power accumulator; and
a control circuit coupled to the power supply and the power accumulator, wherein the control circuit is to:
charge the power accumulator;
determine that a parameter is below a threshold; and
discharge power from the power accumulator to increase power output of the power supply based on the parameter being below the threshold.

33. The surgical instrument of claim 32, wherein the power accumulator is a vibration system to collect power from vibrations of the surgical instrument, the vibration system comprising:
a first electrode;
a second electrode; and
a piezoelectric material disposed between the first and second electrode.

34. The surgical instrument of claim 32, wherein the power accumulator comprises an inductor coiled around a drive bar to harvest energy from oscillatory movement of a drive bar and store the harvest energy in the inductor.

35. The surgical instrument of claim 32, wherein the power accumulator is a boost circuit comprising a variable resistor to adjust an output voltage of the boost circuit.
